(19) United States
(12) Patent Application Publication
(10) Pub. No.: US 2006/0190086 A1
(43) Pub. Date: Aug. 24, 2006

(54) Knee implant

(75) Inventors: Alastair J.T. Clemow, Princeton, NJ (US); Dana C. Mears, Pittsburgh, PA (US)

Correspondence Address: FOLEY AND LARDNER LLP;SUITE 500, 3000 K STREET NW, WASHINGTON, DC, 20007, US

(73) Assignee: MAKO Surgical Corporation

(21) Appl. No.: 11/321,741

(22) Filed: Dec. 30, 2005

Related U.S. Application Data
(60) Provisional application No. 60/655,013, filed on Feb. 22, 2005.

(51) Int. Cl. A61F 2/38

(52) U.S. Cl. 623/20.15; 623/20.3; 623/20.28

(57) ABSTRACT

A modular prosthetic device is provided for replacement of the knee. The device is assembled from a plurality of components, each of which can be inserted through a small incision. After inserting the components through the incision, the device can be assembled within the knee cavity. The modularity of the device enables a surgeon to replace only those regions of the knee that are diseased or damaged, thereby avoiding a complete knee replacement. If, at a later time, additional regions of the knee become diseased or damaged, those additional regions of the knee can be replaced by additional device components and those additional components can be connected to the previously implanted components. By replacing only those regions of the knee that are diseased or damaged and by implanting each of the components through the small incision, the surgery is minimally invasive and, therefore, requires reduced time for healing and rehabilitation.

### CROSS REFERENCE TO RELATED APPLICATIONS

This application claims priority from U.S. Provisional Patent Application Ser. No. 60/655,013, filed Feb. 22, 2005, which is incorporated by reference herein.

### BACKGROUND OF THE INVENTION

This invention relates to prosthetic devices for use in partial or complete knee replacement and associated methods.

Partial or complete replacement of diseased and/or damaged knees with suitable prostheses has become a common surgical procedure. The outcome of such surgery has been found to be favorable in most cases, and the surgery has come to be regarded as a very favorable surgical intervention for restoring function to knees damaged by trauma or degenerative disease. Each year more than 650,000 patients worldwide undergo operations in which either part or all of a knee joint is replaced by an implant, which typically operates well for 10 or more years.

Traditional implant designs include a tibial component and a femoral component, which bears on the tibial component. The femoral component, which is typically made from a cobalt-based alloy, replaces the bearing surfaces of the femur. The tibial component, which is typically a combination of a metallic portion (which is positioned against the bone) and an ultra-high molecular weight polyethylene (“UHMWPE”) portion (which acts as a bearing surface), is implanted upon the proximal end of the tibia. Additionally, a second polyethylene implant may be used to replace the undersurface of the patella so that it slides upon the central portion of the metallic femoral implant. To minimize the problem of wear in the joints, the metallic femoral component is generally polished to a very fine mirrored surface and its bearing surfaces are designed with a sufficient degree of conformity to reduce contact stresses while allowing enough laxity to allow free movement.

A problem with a conventional implant procedure is that the components are relatively large. Even if a component is formed by assembling smaller parts, often those parts need to be assembled before insertion into the patient's body. Consequently, the components must be inserted through relatively long incisions, e.g., three or more inches. For example, the femoral component may be about four inches wide and about three inches high, thereby requiring a correspondingly large incision for implantation. Such large incisions tend to disrupt the tissues associated with the joint and its joint capsule, thereby requiring long healing and rehabilitation periods. As a result, the patient can not quickly return to normal activities. Although surgeons have recognized the desirability of minimizing the size of the incisions, the large size of the prostheses of current designs have frustrated attempts to use smaller incisions, e.g., 1-2 inches in length.

U.S. Published Patent Application No. 2003/0158606, to Coon et al., discloses a knee arthroplasty prosthesis in which a femoral component of a total knee joint replacement is made in multiple pieces, which are inserted separately and assembled within the surgical site. The separate pieces of Coon's femoral component are assembled using mating surfaces generally that are formed at an angle to a plane oriented in an anterior-posterior direction and proximal-distal direction with respect to the femur.

Coon's prosthesis presents a number of shortcomings. For example, Coon's multi-piece prosthesis is disclosed as requiring a three-inch surgical incision. Further, when it is used to manage arthritis of the anterior and medial compartments (or the anterior and lateral compartments), Coon's prosthesis creates an abrupt transition on the lateral (or medial) compartment, thereby creating an interface of metallic implant and adjacent bone; this abrupt transition may promote degeneration in the non-implant region. Moreover, the implant is excessively large with respect to the central portion of the knee.

Another presently known implant prosthesis includes a femoral component for a knee compartment that is assembled from multiple pieces. The femoral component can be assembled from anterior and posterior parts that connect along a lateral-to-medial plane. Such a prosthetic device is not likely to provide long-term durability. For example, as a result of the orientation of the femoral component parts, long-term cyclical loading on the component may cause the component to break.

Another problem associated with a conventional implant procedure is that it may require displacement of significant amounts of healthy bone. For example, a total knee prosthesis may be implanted even if only the medial and anterior compartments of the knee are diseased or damaged. In such a case, even if only the medial region of the femur must be replaced, the healthy lateral region of the femur also will be replaced to accommodate the total knee prosthesis.

Another problem with a conventional implant procedure is that the knee prosthesis may not be sufficiently customizable to meet the optimal needs of individual patients. Although the anatomy of the knee is generally consistent in the sense that it typically includes a femur, tibia, patella, etc., the particular dimensions of the knee structure can differ from patient to patient. For example, if a patient possesses a large medial compartment and a small lateral compartment, a single size femoral implant may be appropriately sized for one of the compartments and inappropriately sized for the other compartment. Moreover, needs can differ from patient to patient based on other factors, such as the extent of knee damage. While custom implants, designed specifically for a given patient, are available from most manufacturers, the time, expense and logistical difficulties in using such implants means that most surgeons will attempt to use “off-the-shelf” prostheses. Consequently some patients receive less than optimum devices.

Another problem is that a conventional knee prosthesis may not be configured to accommodate later surgical procedures. For example, if the attachment of a conventional total knee prosthesis to the bone becomes loose at only one region, often the entire prosthesis will need to be removed and replaced in a later surgical procedure. As another example, if there is an increase in the diseased or damaged area of the knee, a conventional knee prosthesis may need to be removed and replaced with another knee prosthesis in a later surgical procedure. As the removal of a prosthesis reduces the probable life-span of the replacement prosthesis, this is a less than desirable approach. The life-span of the replacement prosthesis is reduced because the removal of original device often requires removal of bone (or damage to the bone) attached to the device. As a result, the replacement device must be correspondingly larger to compensate for the lost or damage bone. Likewise, the surgical incision must be correspondingly larger to accommodate the larger device, thereby prolonging the recovery period. Moreover, the larger replacement device may be more elaborate and/or more expensive than the originally implanted device and/or may compromise the functional result of the procedure.

In light of the foregoing, a need exists for improved prosthetic devices and associated methods.

### SUMMARY OF THE INVENTION

An embodiment of the present invention relates to a method of implanting a prosthetic device. This method includes, among other possible steps: selecting a first side femoral component configured to be implanted on at least one of a lateral condyle and a medial condyle of a femur; implanting the first side femoral component on one of the lateral condyle and the medial condyle of the femur; selecting a second side femoral component configured to be implanted on at least the other of the lateral condyle and the medial condyle of the femur, wherein the second side femoral component is selected from a plurality of femoral components configured to be used with the first side femoral component based on characteristics of the second side femoral component; and implanting the second side femoral component on the femur.

Another embodiment of the present invention relates to a method of implanting a prosthetic device. This method includes, among other possible steps: selecting a first side femoral component configured to be implanted on at least one of a lateral condyle and a medial condyle of a femur; implanting the first side femoral component on one of the lateral condyle and the medial condyle of the femur; selecting a center femoral component configured to be implanted on a central region of the femur, wherein the center femoral component is selected from a plurality of femoral components configured to be used with the first side femoral component based on characteristics of the center femoral component; and implanting the center femoral component on the femur.

Another embodiment of the present invention relates to a method of implanting a prosthetic device. This method includes, among other possible steps: selecting a first side tibial component configured to be implanted on at least one of a lateral region and a medial region of a tibia; implanting the first side tibial component on one of the lateral region and the medial region of the tibia; selecting a second side tibial component configured to be implanted on at least the other of the lateral region and the medial region of the tibia, wherein the second side tibial component is selected from a plurality of tibial components configured to be used with the first side tibial component based on characteristics of the second side tibial component; and implanting the second side tibial component on the tibia.

Another embodiment of the present invention relates to a method of implanting a prosthetic device. This method includes, among other possible steps: selecting a first side tibial component configured to be implanted on at least one of a lateral region and a medial region of a tibia; implanting the first side tibial component on one of the lateral region and the medial region of the tibia; selecting a center tibial component configured to be implanted on a central region of the tibia, wherein the center tibial component is selected from a plurality of tibial components configured to be used with the first side tibial component based on characteristics of the center tibial component; and implanting the center tibial component on the tibia.

Another embodiment of the present invention relates to a prosthetic device, which includes, among other possible things: a first side femoral component configured to be implanted on at least one of a lateral condyle and a medial condyle of a femur; a second side femoral component configured to be implanted on at least the other of the lateral condyle and the medial condyle of the femur, wherein the second side femoral component is selected from a plurality of femoral components configured to be used with the first side femoral component based on characteristics of the second side femoral component.

Another embodiment of the present invention relates to a prosthetic device, which includes, among other possible things: a first side femoral component configured to be implanted on at least one of a lateral condyle and a medial condyle of a femur; and a center femoral component configured to be implanted on a central region of the femur, wherein the center femoral component is selected from a plurality of femoral components configured to be used with the first side femoral component based on characteristics of the center femoral component.

Another embodiment of the present invention relates to a prosthetic device, which includes, among other possible things: a first side tibial component configured to be implanted on at least one of a lateral region and a medial region of a tibia; and a second side tibial component configured to be implanted on at least the other of the lateral region and the medial region of the tibia.

Another embodiment of the present invention relates to a prosthetic device, which includes, among other possible things: a first side tibial component configured to be implanted on at least one of a lateral region and a medial region of a tibia; and a center tibial component configured to be implanted on a central region of the tibia, wherein the center tibial component is selected from a plurality of tibial components configured to be used with the first side tibial component based on characteristics of the center tibial component.

Another embodiment of the present invention relates to a collection of components for forming a prosthetic device. This collection includes, among other possible things: a plurality of first side femoral components configured to be implanted on at least one of a lateral condyle and a medial condyle of a femur and having different characteristics; and a plurality of second side femoral component configured to be implanted on at least the other of the lateral condyle and the medial condyle of the femur and having different characteristics, wherein the second side femoral components can be used with the first side femoral components.

Another embodiment of the present invention relates to a collection of components for forming a prosthetic device. This collection includes, among other possible things: a plurality of first side femoral components configured to be implanted on at least one of a lateral condyle and a medial condyle of a femur and having different characteristics; and a plurality of center femoral components configured to be implanted on a central region of the femur and having different characteristics, wherein the center femoral components can be used with the first side femoral components.

Another embodiment of the present invention relates to a collection of components for forming a prosthetic device. This collection includes, among other possible things: a plurality of first side tibial components configured to be implanted on at least one of a lateral region and a medial region of a tibia and having different characteristics; and a plurality of second side tibial component configured to be implanted on at least the other of the lateral region and the medial region of the tibia and having different characteristics, wherein the second side tibial components can be used with the first side tibial components.

Another embodiment of the present invention relates to a collection of components for forming a prosthetic device. This collection includes, among other possible things: a plurality of first side tibial components configured to be implanted on at least one of a lateral region and a medial region of a tibia and having different characteristics; and a plurality of center tibial components configured to be implanted on a central region of the tibia and having different characteristics, wherein the center tibial components can be used with the first side tibial components.

Another embodiment of the present invention relates to a method of implanting a prosthetic device. This method includes, among other possible steps: evaluating a knee of a patient including a previously implanted prosthetic device; implanting in the knee an additional component of a prosthetic device adjacent the previously implanted prosthetic device, while maintaining in the knee at least a portion of the previously implanted prosthetic device; and attaching the additional component to the maintained portion of the previously implanted prosthetic device.

These and other features, aspects, and advantages of the present invention will become more apparent from the following description, appended claims, and accompanying exemplary embodiments shown in the drawings.

### BRIEF DESCRIPTION OF THE DRAWINGS

FIG. 1 is a front view of a knee joint that includes a lower end of a femur, an upper end of a tibia, a patella (displaced for ease of illustration), and an upper end of a fibula, wherein regions of the knee joint are labeled for ease of explanation;

FIG. 2 is a front view of the knee joint of FIG. 1, with bone removed from medial regions to permit implantation and with ligaments and the patella removed for ease of viewing;

FIGS. 3A, 3B, 3C, and 3D are perspective, top, side, and front views, respectively, of an embodiment of a unicompartmental side femoral component, which may be implanted in the medial region and/or the lateral region of the femur;

FIGS. 4A, 4B, 4C, and 4D are perspective, top, side, and front views, respectively, of an embodiment of a center femoral component, which may be implanted in a position between the medial and lateral regions of the femur;

FIGS. 5A, 5B, 5C, and 5D are perspective, top, side, and front views, respectively, of an embodiment of a unicompartmental side tibial component, which may be implanted in the medial and/or lateral region of the tibia;

FIGS. 6A, 6B, 6C, and 6D are perspective, top, side, and front views, respectively, of an embodiment of a center tibial component, which is configured to be implanted in conjunction with the unicompartmental side tibial component shown in FIGS. 5A-5D;

FIGS. 7A, 7B, 7C, and 7D are perspective, top, side, and front views, respectively, of a first embodiment of a backing tray, which may be implanted in the medial and/or lateral region of the tibia;

FIGS. 8A, 8B, 8C, and 8D are perspective, top, side, and front views, respectively, of an embodiment of a middle backing tray, which is configured to be implanted in conjunction with the backing tray shown in FIGS. 7A-7D;

FIGS. 9A, 9B, 9C, and 9D are perspective, top, side, and front views, respectively, of a second embodiment of a backing tray, which may be implanted in: (i) medial and central regions of the tibia; and/or (ii) central and lateral regions of the tibia;

FIGS. 10A, 10B, 10C, and 10D are perspective, top, side, and front views, respectively, of an embodiment of a half-span tibial component, which may be implanted in (i) medial and central regions of the tibia; and/or (ii) central and lateral regions of the tibia;

FIGS. 11A, 11B, 11C, and 11D are perspective, top, side, and front views, respectively, of an embodiment of a full-span tibial component, which may be implanted in the medial, central, and lateral regions of the tibia;

FIGS. 12A, 12B, 12C, and 12D are perspective, top, side, and front views, respectively, of an embodiment of a tibial component having a posterior cruciate ligament substituting device;

FIGS. 13A, 13B, 13C, and 13D are perspective, top, side, and front views, respectively, of an alternate embodiment of a center tibial component;

FIGS. 14A, 14B, 14C, and 14D are perspective, top, side, and front views, respectively, of an alternate embodiment of a center femoral component;

FIGS. 15A, 15B, 15C, and 15D are perspective, top, side, and front views, respectively, of an embodiment of a patellar backing device, which is configured to be implanted in the back side of the patella;

FIGS. 16A, 16B, 16C, and 16D are perspective, top, side, and front views, respectively, of an embodiment of a tibial tray post, which is configured to be implanted in the tibia;

FIGS. 17A, 17B, 17C, and 17D are perspective, top, side, and front views, respectively, of an embodiment of a half-span femoral component, which is configured to be implanted in the medial region and/or the lateral region of the femur;

FIGS. 18A, 18B, and 18C are exploded front, side, and perspective views, respectively, of a prosthetic device that includes: (a) two femoral components of the type shown in FIGS. 3A-3D for implantation in the medial and lateral regions of the femur; (b) a center femoral component of the type shown in FIGS. 4A-4D for implantation between the femoral components; (c) two backing trays of the type shown in FIGS. 7A-7D for implantation in the medial and lateral regions of the tibia; (d) a middle backing tray of the type shown in FIGS. 8A-8D for implantation in the central region of the tibia between the backing trays; (e) two tibial components of the type shown in FIGS. 5A-5D for implantation in the backing trays; (f) a center tibial component of the type shown in FIGS. 6A-6D for implantation in the middle backing tray; and (g) a plurality of tibial tray posts of the type shown in FIGS. 16A-16D for implantation in the lateral, central, and medial regions of the tibia;

FIGS. 19A, 19B, and 19C are exploded front, side, and perspective views, respectively, of a prosthetic device that includes: (a) two femoral components of the type shown in FIGS. 3A-3D for implantation in the medial and lateral regions of the femur; (b) two backing trays of the type shown in FIGS. 7A-7D for implantation in the medial and lateral regions of the tibia; (c) a middle backing tray of the type shown in FIGS. 8A-8D for implantation in the central region of tibia between the backing trays; (d) two tibial components of the type shown in FIGS. 5A-5D for implantation in the backing trays; (e) a cruciate substituting center femoral component of the type shown in FIGS. 13A-13D for implantation in the central region of the femur between the femoral components in the femur; (f) a center tibial component of the type shown in FIGS. 13A-13D for implantation in the middle backing tray; and (g) a plurality of tibial tray posts of the type shown in FIGS. 16A-16D for implantation in the lateral, central, and medial regions of the tibia;

FIGS. 20A, 20B, 20C, and 20D are exploded front, exploded side, exploded perspective, and assembled views, respectively, of a prosthetic device that includes: (a) two femoral components of the type shown in FIGS. 17A-17D for implantation in the medial, central, and lateral regions of the femur; (b) two backing trays of the type shown in FIGS. 7A-7D for implantation in the medial and lateral regions of the tibia; (c) a middle backing tray of the type shown in FIGS. 8A-8D for implantation in the central region of the tibia between the backing trays; (d) two tibial components of the type shown in FIGS. 5A-5D for implantation in the backing trays; (e) a center tibial component of the type shown in FIGS. 6A-6D for implantation in the middle backing tray; and (f) a plurality of tibial tray posts of the type shown in FIGS. 16A-16D for implantation in the lateral, central, and medial regions of the tibia;

FIG. 21 is a cross-sectional view of the femur with a femoral component of the type shown in FIGS. 3A-3D implanted on the medial region of the femur;

FIG. 22 is a cross-sectional view of the tibia with a backing tray of the type shown in FIGS. 7A-7D implanted in the medial region of the tibia and with a tibial component of the type shown in FIGS. 5A-5D implanted in the backing tray; and

FIG. 23 is a cross-sectional view of the tibia with a tibial component of the type shown in FIGS. 5A-5D implanted directly on the medial region of the tibia.

### DETAILED DESCRIPTION OF PREFERRED EMBODIMENTS

Presently preferred embodiments of the invention are illustrated in the drawings. An effort has been made to use the same or like reference numbers throughout the drawings to refer to the same or like parts.

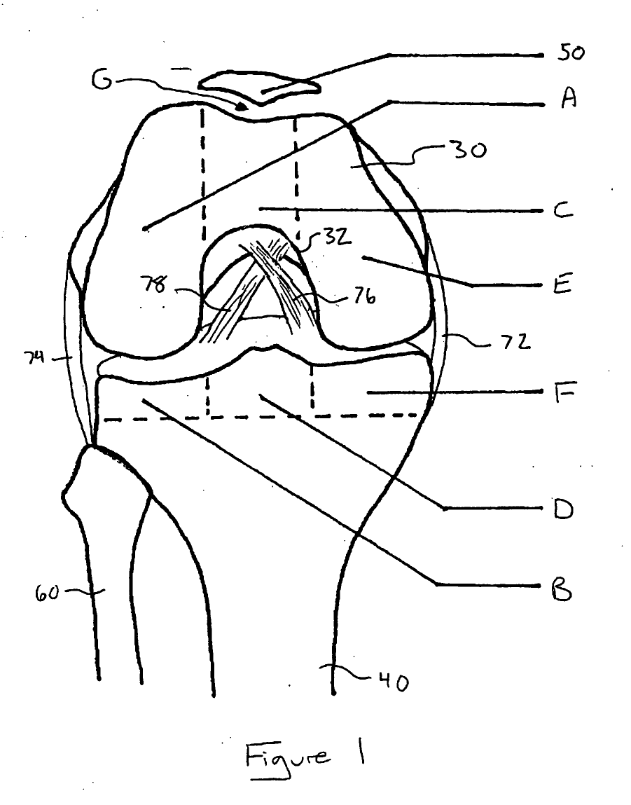

FIG. 1 is a diagram of a knee joint that includes a lower end of a femur 30, an upper end of a tibia 40, a fibula 60, and a patella 50. The patella 50 moves relative to the femur 30 and tibia 40, when the knee joint articulates. The femur 30 is joined to the tibia 40 by a medial collateral ligament (“MCL”) 72, a posterior cruciate ligament (“PCL”) 78, and an anterior cruciate ligament (“ACL”) 76. The femur 30 is joined to the fibula 60 by a lateral collateral ligament (“LCL”) 74.

The lower end of the femur 30 is conceptually divided into a lateral (i.e., outside) condyle region A, a central region C (which contains a patellar groove 32 having an inverted U-shape), and a medial condyle (i.e., inside) region E. Similarly, the upper end of the tibia 40 is also conceptually divided into lateral B, central D, and medial F regions, which correspond, respectively, to the lateral A, central C, and medial E regions of the femur 30. Finally, the space between the patella 50 and the femur 30 or the tibia 40 (depending on the bending state of the leg) defines a patellar region G.

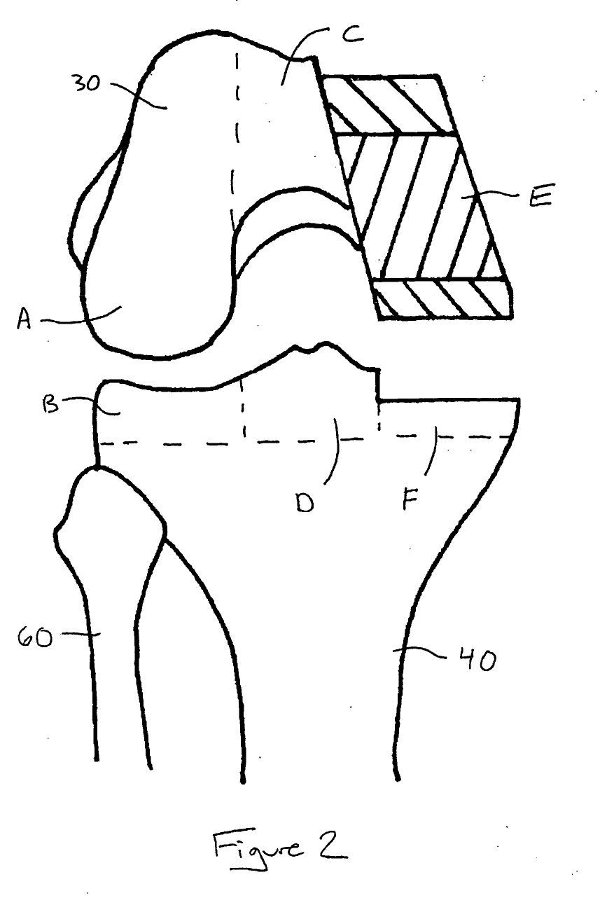

FIG. 2 is a front view of the knee joint of FIG. 1, with bone removed from medial regions E and F to facilitate implantation of a prosthetic device. For example, a unicompartmental side femoral component 100 can be implanted in region E, and a unicompartmental side tibial component 300 can be implanted in region F.

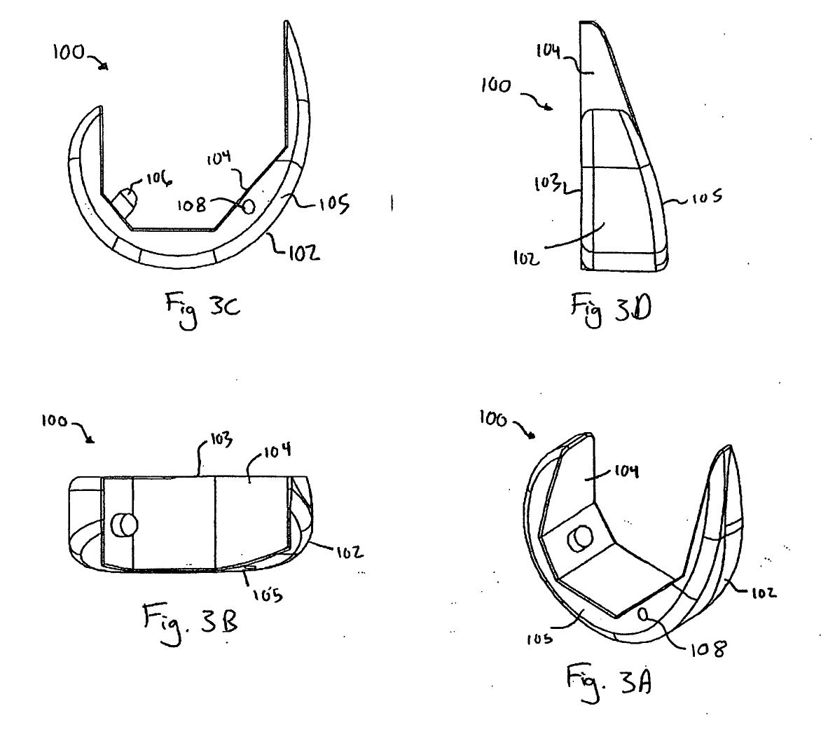

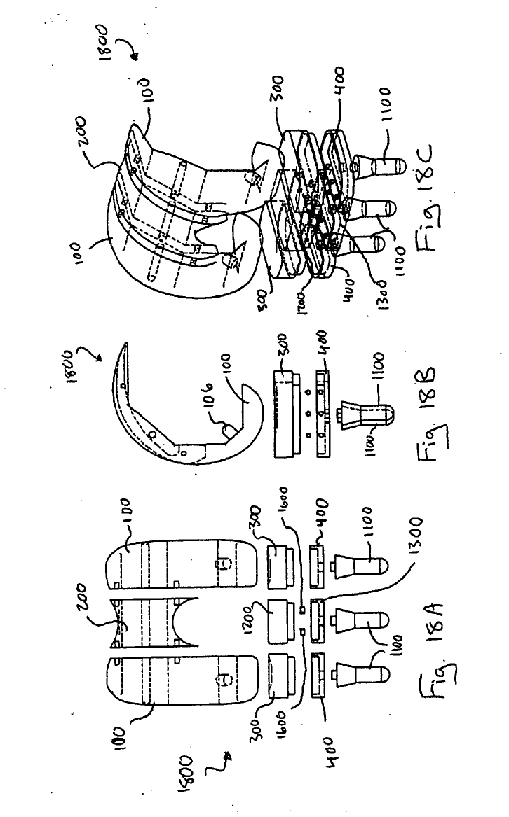

An embodiment of a side femoral component 100 is shown in FIGS. 3A-3D. The particular embodiment shown is configured for insertion into medial femoral region E. A mirror image (shown in FIGS. 18A-18C) of the femoral component 100 can also be implanted in lateral femoral region A. Preferably, the femoral component 100 is formed of a strong biocompatible metal such as a cobalt-chromium alloy, a titanium alloy, or stainless steel. Additionally or alternatively, the component may be formed from a strong ceramic (e.g., an alumina, zirconia, or carbon-based ceramic), one or more high performance polymers, and/or one or more high performance polymer composites (e.g., a composite material made of nano particles of polytetrafluoroethylene (“PTFE”) and polyetheretherketone (“PEEK”); particle ratios can be either fixed or can vary in the range on 0-100% (and vice versa), thereby enabling gradual changes in material properties in the component). Additionally or alternatively, the component may be made of a material of the types described in U.S. patent application Ser. No. 10/914,615, which was filed Aug. 9, 2004, which is entitled “Low Friction And Low Wear Polymer/Polymer Composites”, and which is incorporated herein by reference in its entirety. Of course, other biocompatible materials may be used to form the component 100.

The femoral component 100 is generally c-shaped and includes a front side 102, a right face 103, a rear side 104, and a left face 105. The front side 102 is generally smooth and curved such that the front side 102 can engage the underside of the patella 50 and the upper end of the tibia 40. The rear side 104 includes a projection 106 that is configured to be journalled into a corresponding hole formed (e.g., by drilling) in the femur 30. The projection 106 serves as a stabilizing unit of the component 100 when implanted in the femur 30. The femoral component 100 can be further stabilized by use of, for example, bone cement, a porous bone ingrowth surface or an outgrowth material (e.g., a cobalt-chromium alloy, a titanium alloy, a superficial ceramic coating, etc.), both of which will facilitate bone growth around the component 100, etc.

The femoral component 100 may include a connecting mechanism (e.g., screws, morse tapers, dovetail tenon/mortise, locking clips, etc.) to connect it to adjacent components. In the illustrated embodiment, the connecting mechanism includes holes 108, which are provided on the right and left faces 103, 105 and which are sized to receive mating pins (which may be similar to pins 1600 shown in FIGS. 18A and 18B).

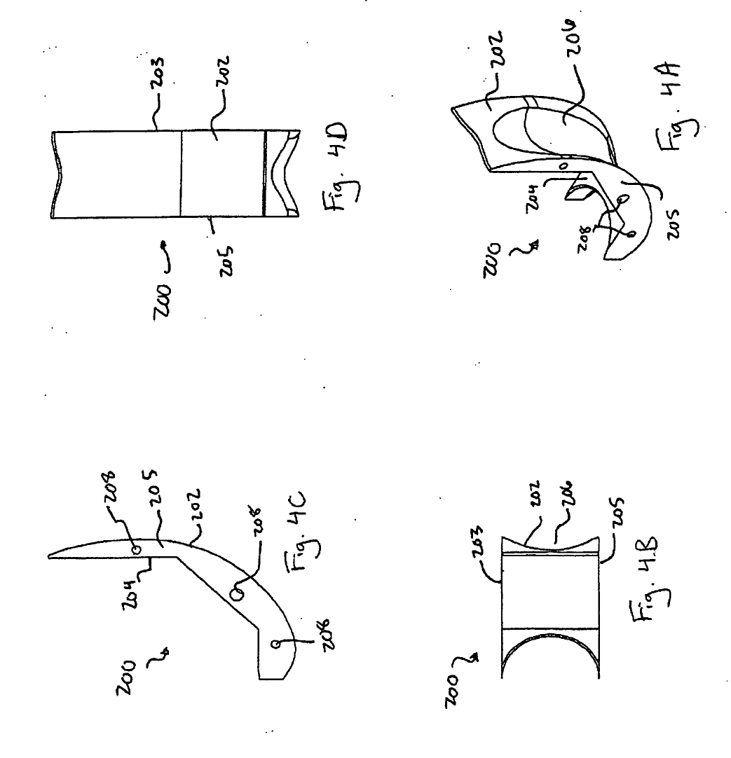

FIGS. 4A-4D depict an embodiment of a center femoral component 200 (or patellar groove component). The center femoral component 200 is configured to be implanted in the patellar groove 32, between the medial E and lateral A regions of the femur 30. The center femoral component is configured to be used with the side femoral component 100. Preferably, the center femoral component 200 is formed of a strong biocompatible metal such as cobalt-chromium alloy, a titanium alloy, or stainless steel. Additionally or alternatively, the component may be formed from a strong ceramic (e.g., an alumina, zirconia, or carbon-based ceramic), one or more high performance polymers, and/or one or more high performance polymer composites (e.g., a composite material made of nano particles of PTFE and PEEK; particle ratios can be either fixed or can vary in the range on 0-100% (and vice versa), thereby enabling gradual changes in material properties in the component). Additionally or alternatively, the component may be made of a material of the types described in U.S. patent application Ser. No. 10/914,615. Of course, other biocompatible materials may be used to form the component 200. This center component 200 does not need to be composed of the same material as either or both of the lateral and medial components 100 of the femur 30.

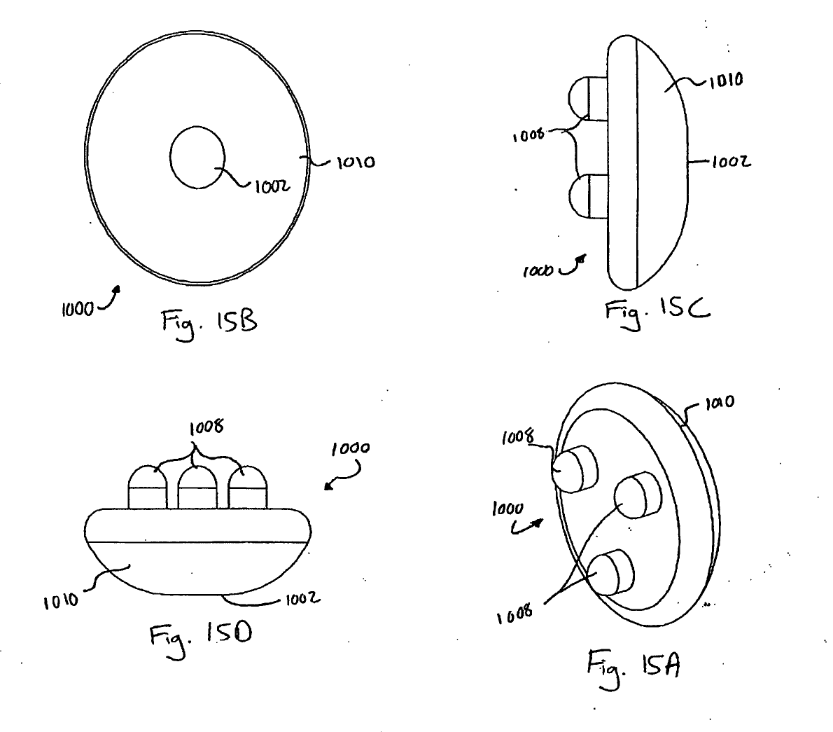

Similar to the side femoral component 100, the center femoral component 200 is generally c-shaped. In addition, the center femoral component 200 includes a curved front side 202, a left face 203, a rear side 204, and a right face 205. The front face 202 includes a depression 206, the importance of which will later be described with respect to a patellar backing device 1000 shown in FIGS. 15A-15D.

The center femoral component 200, like the side femoral component 100, can include a connection mechanism (e.g., screws, morse tapers, dovetail tenon/mortise, locking clips, etc.). In the shown embodiment, the connection mechanism includes holes 208, which are configured to receive pins (which may be similar to pins 1600 shown in FIGS. 18A and 18B). The holes 208 of the center femoral component 200 are provided on both faces 203, 205. As a result, the center femoral component 200 can engage a side femoral component 100 provided on its right face 203, on its left face 205, or both.

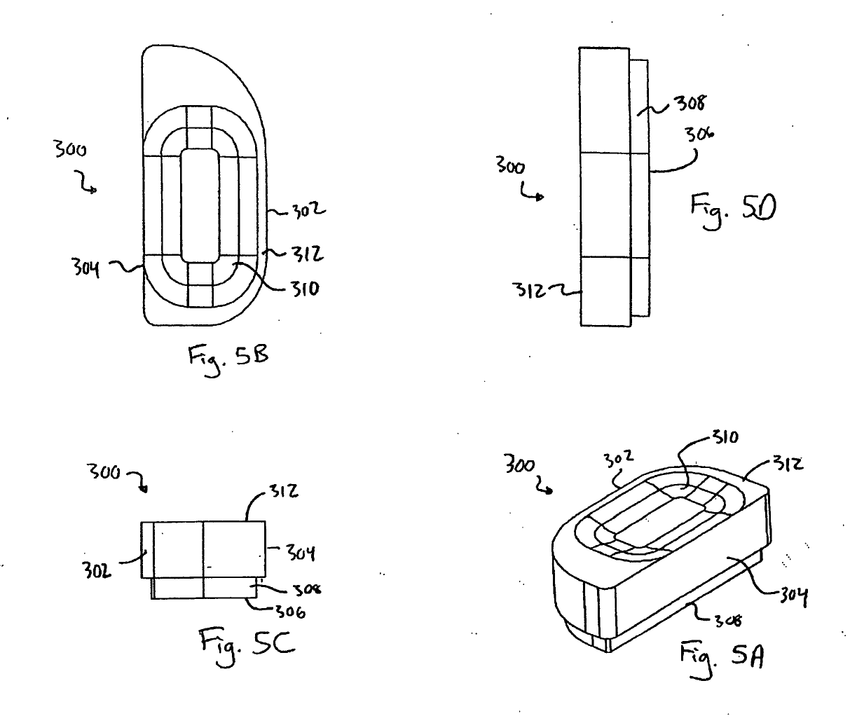

FIGS. 5A, 5B, 5C, and 5D are perspective, top, side, and front views, respectively, of an embodiment of side tibial component 300 (or end support). The side tibial component 300 is configured to be implanted in the medial region F of the tibia 40. A mirror image embodiment (shown in FIGS. 18A-18C) of the side tibial component 300 can be implanted in the lateral region B of the tibia 40. Preferably, the side tibial component 300 is formed of a strong biocompatible metal such as a cobalt-chromium alloy, a titanium alloy, or stainless steel. Additionally or alternatively, the side tibial component 300 may be formed from a strong ceramic (e.g., an alumina, zirconia, or carbon-based ceramic), one or more high performance polymers, and/or one or more high performance polymer composites (e.g., a composite material made of nano particles of PTFE and PEEK; particle ratios can be either fixed or can vary in the range on 0-100% (and vice versa), thereby enabling gradual changes in material properties in the component). Additionally or alternatively, the component may be made of a material of the types described in U.S. patent application Ser. No. 10/914,615. Of course, other biocompatible materials may be used to form the component 300. And, in some embodiments, the side tibial component 300 may be formed of polyethylene.

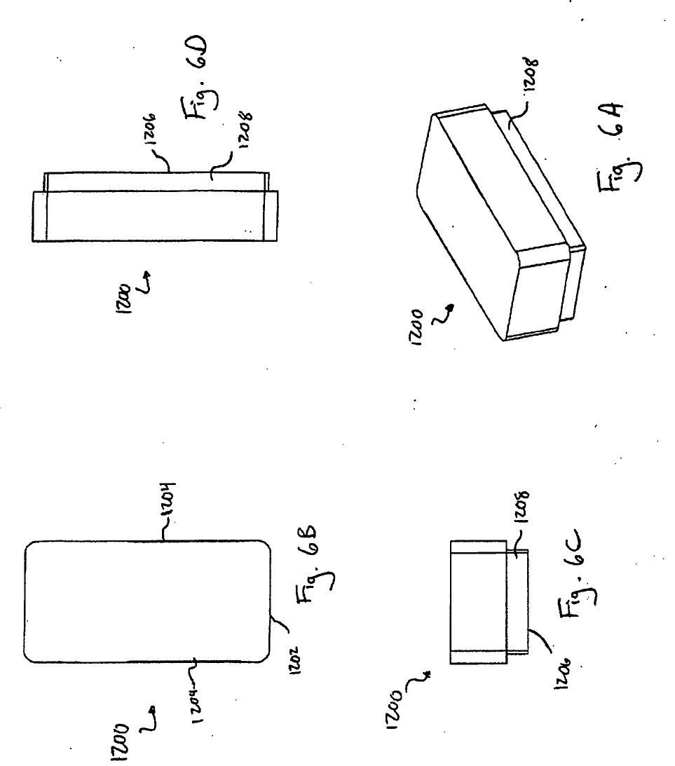

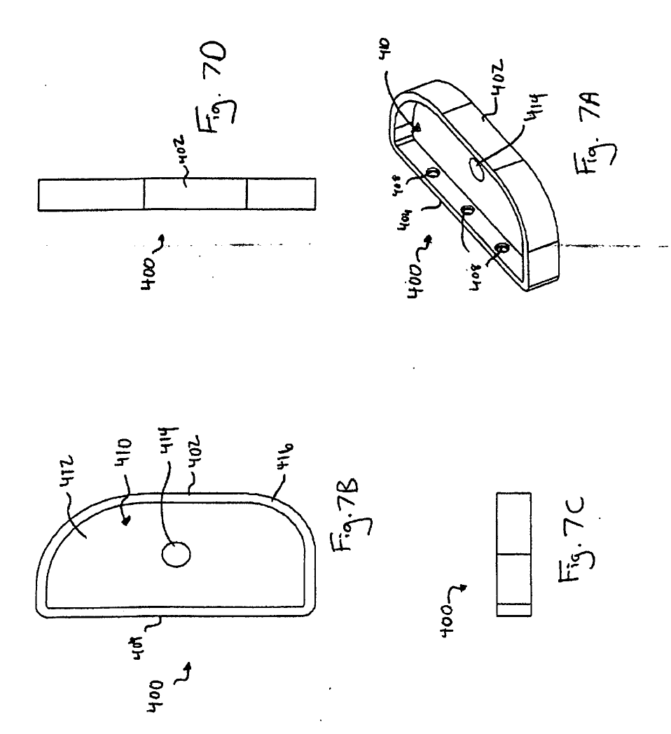

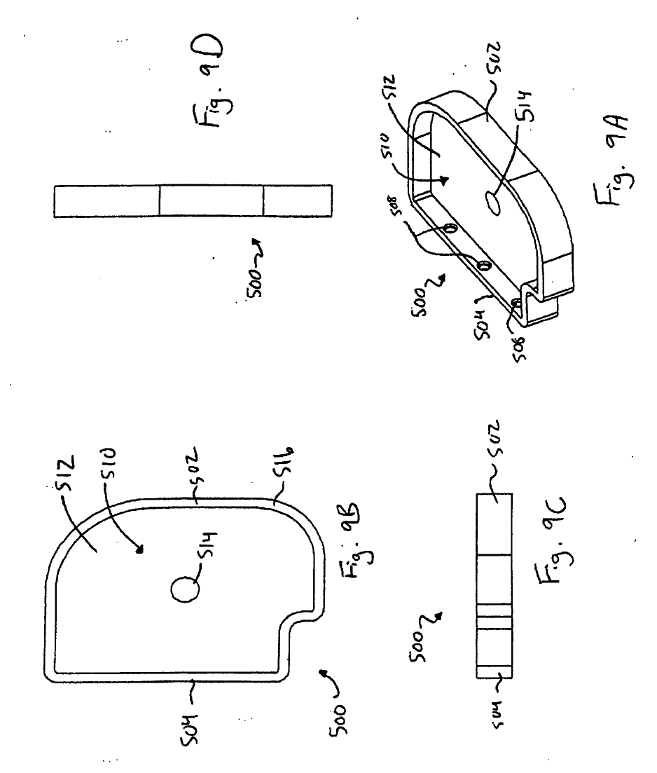

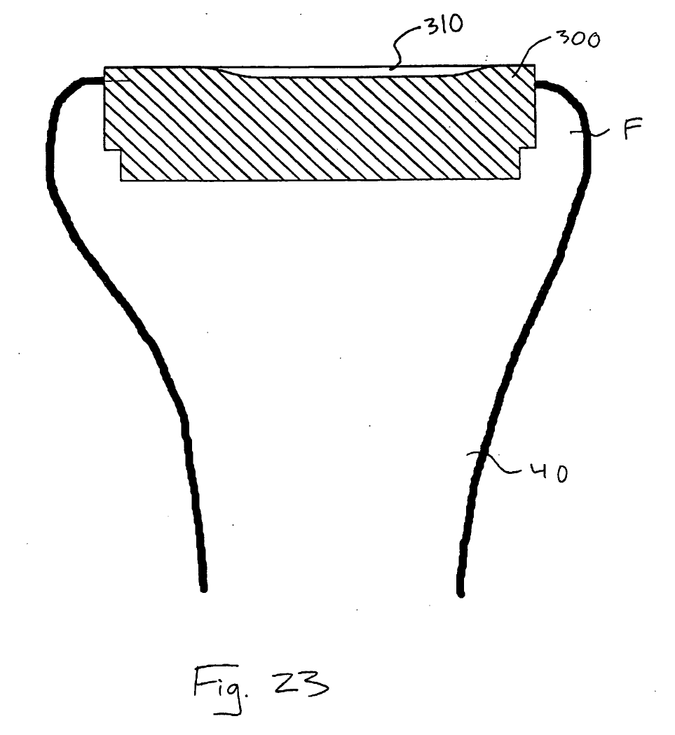

The side tibial component 300 includes a flat, back face 304 and a curved front face 302, which is designed to replicate the curved front and inner surfaces of the medial region F of the tibia 40. In addition, an underside 306 of the side tibial component 300 is formed with a lip 308, which is configured to be implanted directly in the tibia 40 (as shown in FIG. 23) or in a like-sized well 410 formed in backing tray 400, 500 (which is later described in detail with respect to FIGS. 7A-7D and 9A-9D), thereby enhancing the stability of the implantation. A depression 310 is formed in a topside 312 of the side tibial component 300. The depression 310 is configured to engage, for example, the curved front face 102 of the side femoral component 100. Although not shown, the side tibial component 300 may be provided with a connection mechanism (e.g., pins 1600 shown in FIGS. 18A and 18B, screws, morse tapers, dovetail tenon/mortise, locking clips, etc.) that is configured to engage a center tibial component 1200, which will hereafter be discussed with respect to FIGS. 6A-6D.

The center tibial component 1200 (or middle support), which is configured to be implanted in conjunction with the unicompartmental side tibial component 300, can be implanted in the central region D of the tibia 40. Preferably, the center tibial component 1200 is formed of a strong biocompatible metal such as a cobalt-chromium alloy, a titanium alloy, or stainless steel. Additionally or alternatively, the center tibial component 1200 may be formed from a strong ceramic (e.g., an alumina, zirconia, or carbon-based ceramic), one or more high performance polymers, and/or one or more high performance polymer composites (e.g., a composite material made of nano particles of PTFE and PEEK; particle ratios can be either fixed or can vary in the range on 0-100% (and vice versa), thereby enabling gradual changes in material properties in the component). Additionally or alternatively, the component may be made of a material of the types described in U.S. patent application Ser. No. 10/914,615. Of course, other biocompatible materials may be used to form the component 1200. And, in some embodiments, the center tibial component 1200 may be formed of polyethylene.

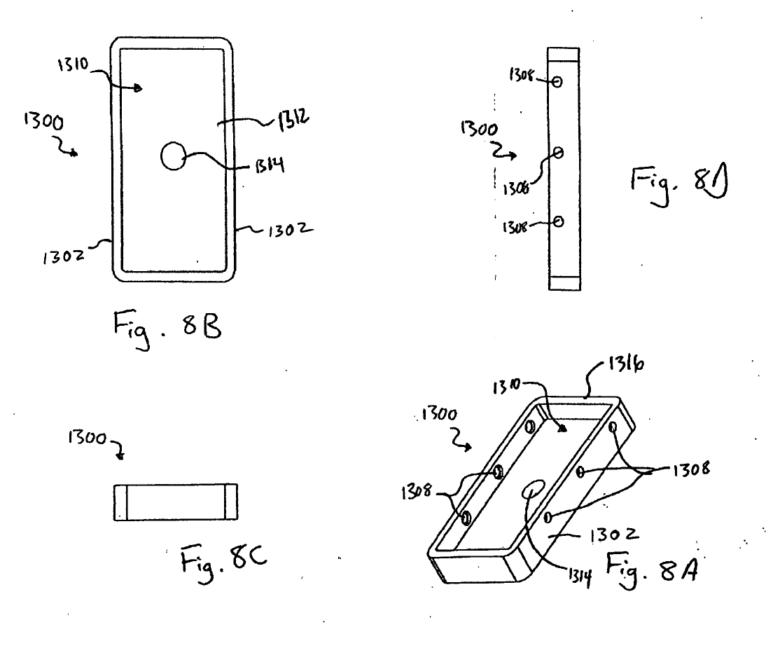

The center tibial component 1200 is generally rectangularly shaped and, like the side tibial component 300, is provided with an underside 1206 having a lip 1208. The lip 1208 of the center tibial component 1200, like the lip 308 of the side tibial component 300, is configured to fit directly in the tibia 40 or in a well 1310 in a middle backing tray 1300 (which is later described in detail with respect to FIGS. 8A-8D), thereby enhancing the stability of the implantation. Longer sides 1204 of the generally rectangularly shaped center tibial component 1200 are sized and configured to rest flush against the flat, back face 304 of the side tibial component 300.

FIGS. 7A-7D are perspective, top, side, and front views, respectively, of a first embodiment of a backing tray 400, which may be implanted in the medial region F of the tibia 40. A mirror image (shown in FIGS. 18A-18C) embodiment of the backing tray 400 can be implanted in the lateral region B of the tibia 40. Preferably, the backing tray 400 is formed of a strong biocompatible metal such as a cobalt-chromium alloy, a titanium alloy, or stainless steel. Additionally or alternatively, the backing tray 400 may be formed from a strong ceramic (e.g., an alumina, zirconia, or carbon-based ceramic), one or more high performance polymers, and/or one or more high performance polymer composites (e.g., a composite material made of nano particles of PTFE and PEEK; particle ratios can be either fixed or can vary in the range on 0-100% (and vice versa), thereby enabling gradual changes in material properties in the component). Additionally or alternatively, the component may be made of a material of the types described in U.S. patent application Ser. No. 10/914,615. Of course, other biocompatible materials may be used to form the tray 400. And, in some embodiments, the backing tray 400 may be formed of polyethylene.

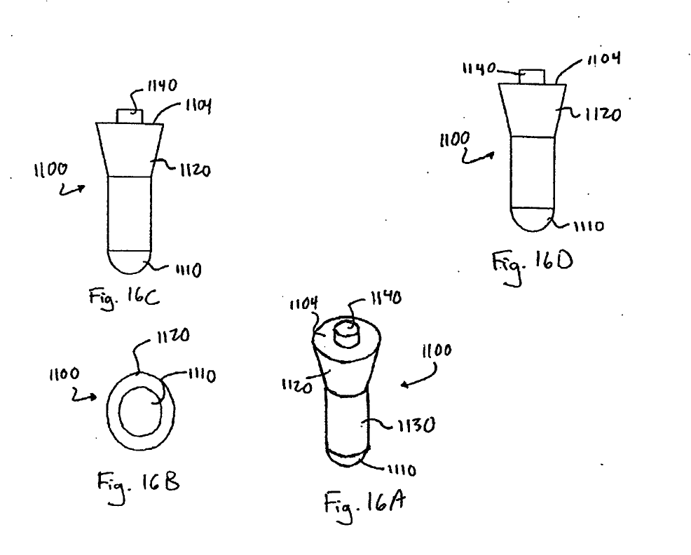

The backing tray 400 tray has a curved outer side 402 and a flat, back wall 404. A well 410, which is defined in the backing tray 400 by a rim 416, may include a connection mechanism along the back wall 404 and/or in a base 412. In the shown embodiment, for example, the backing tray 400 may have a connection mechanism in the form of holes 408 formed in the back wall 404 and a hole 414 formed in the base 412. The hole 414 in the base 412 may, for example, receive a fastener (e.g., a tibial tray post 1100, which is later described with respect to FIGS. 16A-16D) that can be drilled into the tibia 40. In contrast, the holes 408 in the back wall 404 are configured to engage a pin 1600 (shown in FIGS. 18A and 18B) that engages corresponding holes in a middle backing tray 1300, which will now be described with respect to FIGS. 8A-8D.

The middle backing tray 1300, which is configured to be implanted in conjunction with the backing tray 400, is designed to be implanted in the central region D of the tibia 40. Preferably, the middle backing tray 1300 is formed of a strong biocompatible metal such as a cobalt-chromium alloy, a titanium alloy, or stainless steel. Additionally or alternatively, the middle backing tray 1300 may be formed from a strong ceramic (e.g., an alumina, zirconia, or carbon-based ceramic), one or more high performance polymers, and/or one or more high performance polymer composites (e.g., a composite material made of nano particles of PTFE and PEEK; particle ratios can be either fixed or can vary in the range on 0-100% (and vice versa), thereby enabling gradual changes in material properties in the component). Additionally or alternatively, the component may be made of a material of the types described in U.S. patent application Ser. No. 10/914,615. Of course, other biocompatible materials may be used to form the tray 1300. And, in some embodiments, the middle backing tray 1300 may be formed of polyethylene.

Similar to the center tibial component 1200, the middle backing tray 1300 is generally rectangularly shaped. Longer sides 1302 of the generally rectangularly shaped middle backing tray 1300 are sized and configured to rest flush against the flat, back wall 404 of the backing tray 400. Moreover, the longer sides 1302 may have a connection mechanism therein that is configured to engage the flat, back wall 404 of the backing tray 400. For example, the longer sides 1302 are provided with holes 1308, which may engage pins 1600 (shown in FIGS. 18A and 18B) that also engage holes 408 in the back wall 404 of the backing tray 400. Of course, other fasteners (e.g., screws, morse tapers, dovetail tenon/mortise, locking clips, etc.) may be employed. The middle backing tray 1300 also may be provided with a connection mechanism to enhance the implantation of middle backing tray 1300. For example, a connection mechanism (e.g., holes 1314) may be provided in a well 1310, which is defined by a base 1312 surrounded by a rim 1316. The holes 1314 may be configured to receive a tibial tray post 1100, which is later described with respect to FIGS. 16A-16D.

For a bicompartmental knee replacement, two backing trays 400 may be connected on either side of a middle backing tray 1300 and the combination thereof can be implanted in the lateral B, central D, and medial F regions of the tibia 40. In contrast to this three-part implantation, another embodiment of the invention can span the lateral B, central D, and medial F regions of the tibia 40, while being in only two parts. This two-part embodiment may be formed of two individual backing trays 500, which will now be described with respect to FIGS. 9A-9D.

The backing tray 500 may be implanted alone or in combination with a mirror image embodiment (not shown). In the shown embodiment, the backing tray 500 is configured to be implanted in the medial region F and in roughly the medial half of the central region D of the tibia 40. The mirror image embodiment (not shown) is correspondingly configured to be implanted in the lateral region B and in roughly the lateral half of the central region D of the tibia 40. Preferably, the backing tray 500 is formed of a strong biocompatible metal such as a cobalt-chromium alloy, a titanium alloy, or stainless steel. Additionally or alternatively, the backing tray 500 may be formed from a strong ceramic (e.g., an alumina, zirconia, or carbon-based ceramic), one or more high performance polymers, and/or one or more high performance polymer composites (e.g., a composite material made of nano particles of PTFE and PEEK; particle ratios can be either fixed or can vary in the range on 0-100% (and vice versa), thereby enabling gradual changes in material properties in the component). Additionally or alternatively, the component may be made of a material of the types described in U.S. patent application Ser. No. 10/914,615. Of course, other biocompatible materials may be used to form the tray 500.

The backing tray 500 includes a curved outer face 502 and a flat back wall 504, which is configured to rest flat against a similar back wall of the mirror image embodiment. Moreover, the back walls 504 may have a corresponding connection mechanism thereon such as, for example, holes 508. The holes 508 in the shown embodiment may engage pins (which may be similar to pins 1600 shown in FIGS. 18A and 18B) that also engage similar holes protruding from the back wall of the mirror image embodiment. Of course, other fasteners (e.g., screws, morse tapers, dovetail tenon/mortise, locking clips, etc.) may be employed. Finally, like the previous backing tray 400 and middle backing tray 1300, this backing tray 500 embodiment may comprises a well 510, which is defined by a base 512 surrounded by an outer rim 516. Moreover, the base 512 of the well 510 may be provided with a connection mechanism, e.g., a hole 514. The hole 514 may be configured to receive a tibial tray post 1100, which is later described with respect to FIGS. 16A-16D

Regardless of whether the first embodiment backing tray 400 (in conjunction with a middle backing tray 1300) or the second embodiment backing tray 500 is used, both embodiments are configured to support a side tibial component 300 and a center tibial component 1200. Moreover, if two first embodiment backing trays 400 (and a middle backing tray 1300) are combined or if two second embodiment backing trays 500 are combined, the combinations of the backing tray embodiments 400, 1300, 500 are configured to support two side tibial components 300 and a center tibial component 1200 provided between the side tibial components 300.

In this disclosure it is to be understood that when a backing tray is used in conjunction with a tibial component, the backing tray is to be considered a part of the tibial component. In other words, it should be understood that the implanted backing tray(s) and tibial component in sum define a “tibial component.”

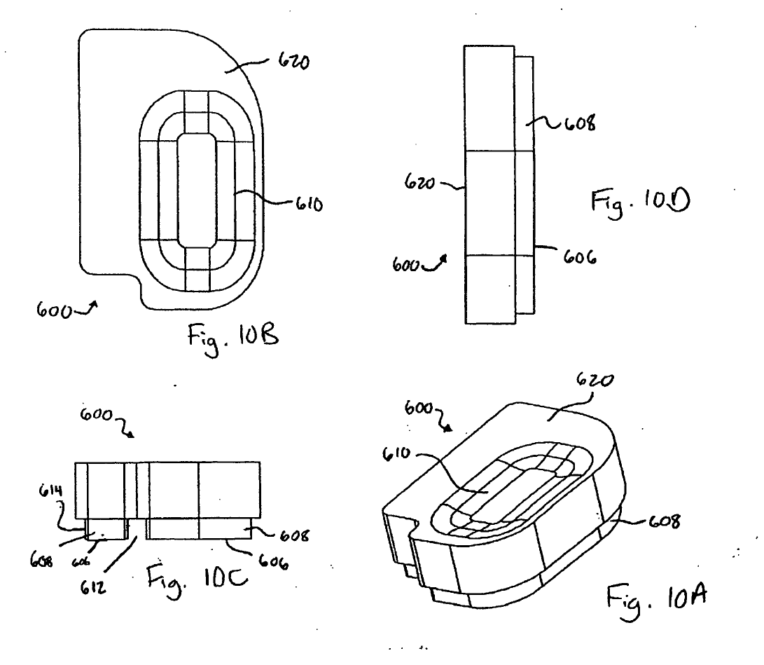

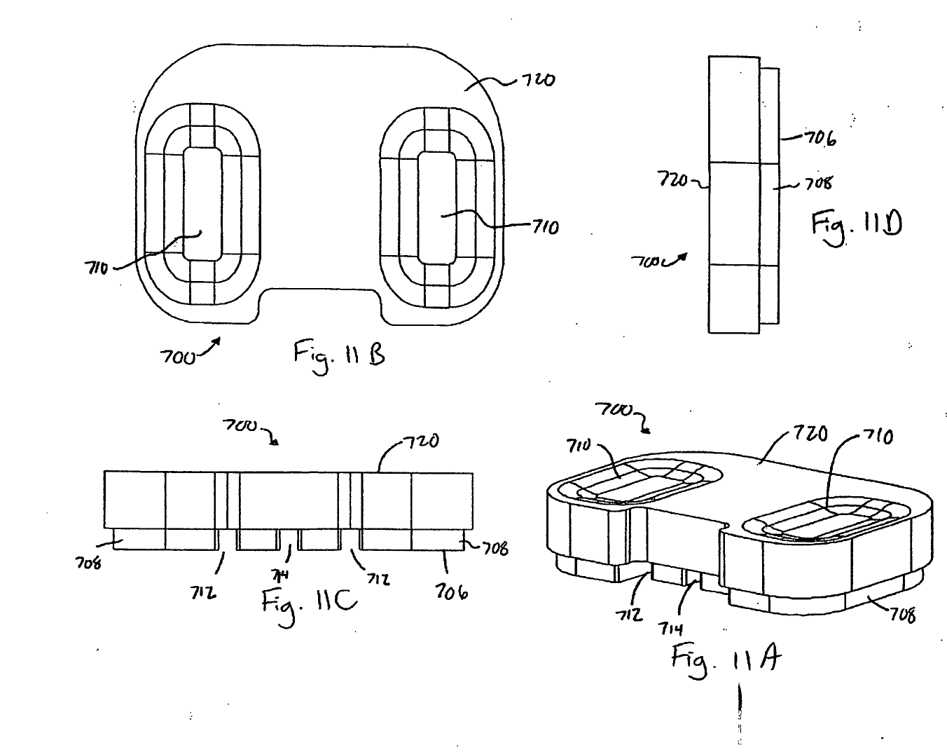

To combine the functionality of the side tibial components 300 and the center tibial component 1200, a surgeon can employ half-span or full-span tibial components 600, 700, which will hereafter be discussed with respect to FIGS. 10A-11D. Preferably, the half-span tibial component 600 and the full-span tibial component 700 are formed of a strong biocompatible metal such as a cobalt-chromium, a titanium alloy, or stainless steel. Additionally or alternatively, the backing components 600, 700 may be formed from a strong ceramic (e.g., an alumina, zirconia, or carbon-based ceramic), one or more high performance polymers, and/or one or more high performance polymer composites (e.g., a composite material made of nano particles of PTFE and PEEK; particle ratios can be either fixed or can vary in the range on 0-100% (and vice versa), thereby enabling gradual changes in material properties in the component). Additionally or alternatively, the component may be made of a material of the types described in U.S. patent application Ser. No. 10/914,615. Of course, other biocompatible materials may be used to form the components 600, 700. Regardless of whether the first embodiment backing tray 400 (in conjunction with a middle backing tray 1300) or the second embodiment backing tray 500 is used, both embodiments are configured to support a half-span tibial component 600, which is hereafter discussed with respect to FIGS. 10A-10D.

The half-span tibial component 600 includes an underside 606 that is circumscribed by a lip 608. The lip 608 is configured to rest on the outer rim 416 of the first embodiment backing tray 400 and the outer rim 516 of the second embodiment backing tray 500. As best shown in FIG. 10C, the underside 606 has a gap 612, which is configured to receive the rim 416 that defines the flat back wall 404 of the first embodiment backing tray 400 and the rim 1316 that defines the longer wall 1312 of the middle backing tray 1300. In contrast, the rim 516 that defines the back wall 504 of the second backing tray 500 embodiment is configured to wrap around a back wall 614 of the lip 608. A depression 610, which is provided in a top side 620 of the half-span tibial component 600, is configured to receive a femoral component 100 of the type shown in FIGS. 3A-3D.

Of course, for a bicompartmental procedure in which two first embodiment backing trays 400 are combined with a middle backing tray 1300, the three-part combination can support the shown half-span tibial component 600 and a mirror image (not shown) embodiment. Similarly, if two second embodiment backing trays 500 are combined, the two-part combination can also support the shown half-span tibial component 600 and the mirror image embodiment thereof.

To eliminate having to use both a half-span tibial component 600 and its mirror image embodiment, the half-span tibial component 600 and the mirror image embodiment can be combined as a full-span tibial component 700, such as that shown in FIGS. 11A-1 ID, which will now be described in detail.

The full-span tibial component 700, like the half-span tibial component 600, has an underside 706 that is provided with a lip 708. In addition, the underside 706 is also provided with gaps 712 that are configured to receive the rims 416, 1316 of the first embodiment backing tray 400 and the middle backing tray 1300. In addition, however, the underside 706 is also provided with another gap 714 that is configured to receive the rims 516 of the flat, back walls 504 of the second embodiment backing trays 500. Finally, depressions 710, which are provided in a top side 720 of the full-span tibial component 700, are configured to receive a femoral components 100 of the type shown in FIGS. 3A-3D.

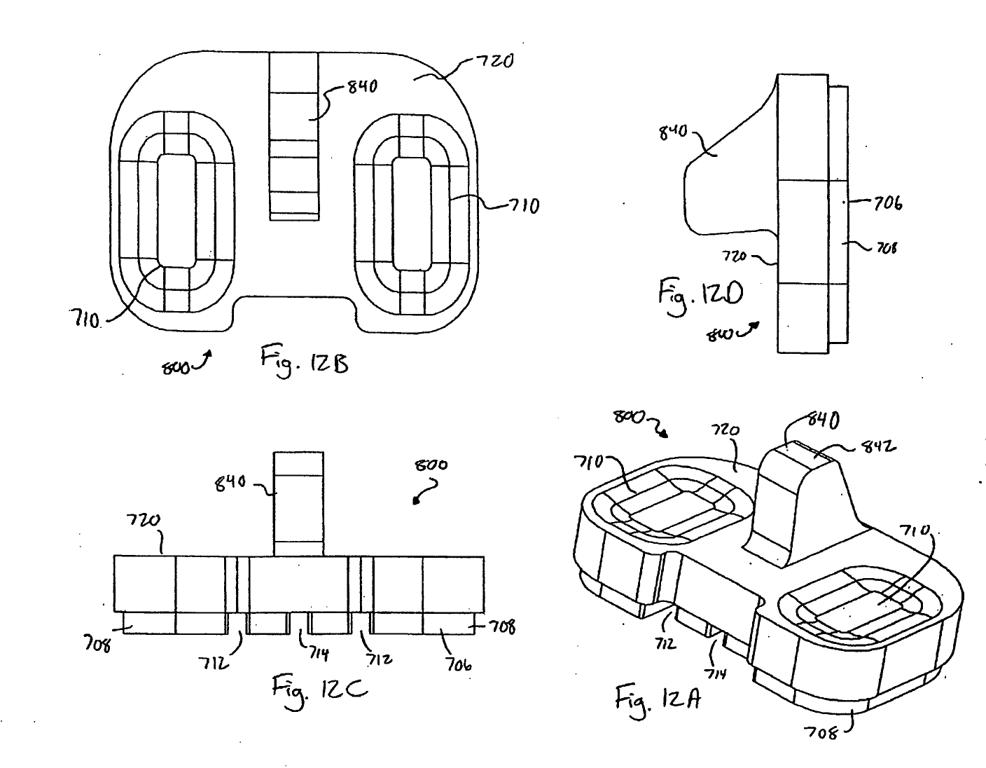

If, during a bicompartmental procedure, the PCL is to be replaced, the surgeon can use a device that enjoys the functionality of the full-span tibial component 700 but provides additional functionality for the replacement of the PCL. An embodiment of such a device is shown in FIGS. 12A-12D, which will hereafter be discussed in detail and which define a first embodiment PCL substituting device 800. Preferably, the PCL substituting device 800 is formed of a strong biocompatible metal such as a cobalt-chromium, a titanium alloy, or stainless steel. Additionally or alternatively, the PCL substituting device 800 may be formed from a strong ceramic (e.g., an alumina, zirconia, or carbon-based ceramic), one or more high performance polymers, and/or one or more high performance polymer composites (e.g., a composite material made of nano particles of PTFE and PEEK; particle ratios can be either fixed or can vary in the range on 0-100% (and vice versa), thereby enabling gradual changes in material properties in the component). Additionally or alternatively, the component may be made of a material of the types described in U.S. patent application Ser. No. 10/914,615. Of course, other biocompatible materials may be used to form the PCL substituting device 800.

But for a PCL replacement fin 840, the PCL substituting device 800 is identical to the full-span tibial component 700 and, therefore, like parts are given like reference numbers, a repetitive discussion of which will be omitted. The fin 840 is configured to fit within the patellar groove 32 and to engage a center femoral component 900, which is later discussed in detail.

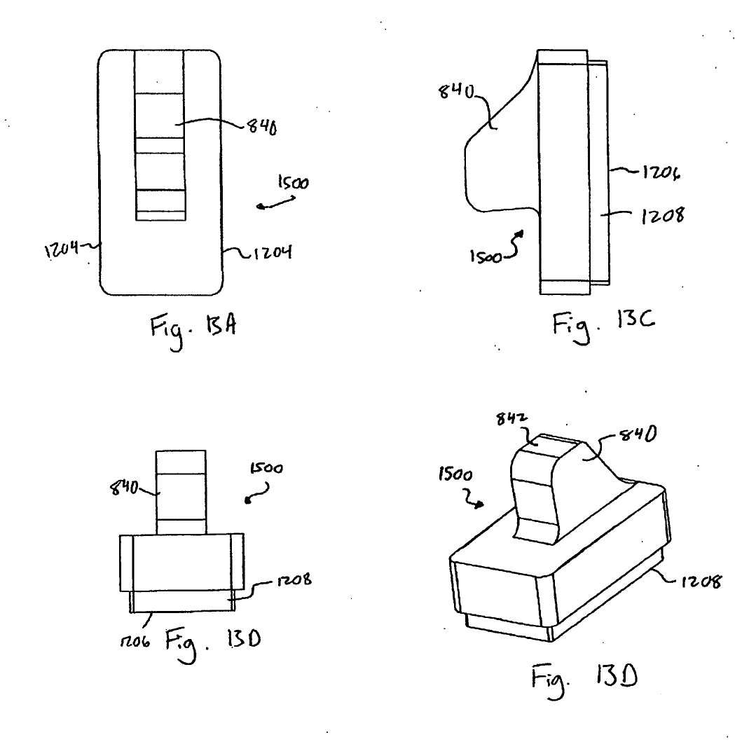

If, after a previous unicompartmental procedure, it becomes necessary to replace the PCL and if the other compartment of the knee remains healthy, the present invention provides a method and apparatus for maintaining that other compartment. To enable such a procedure, the following steps are to be taken. If, during the previous procedure, a first embodiment backing tray 400 was implanted (presumably along with a side tibial component 300), a middle backing tray 1300 is to be implanted in the central region D of the tibia 40. If, instead, a second embodiment backing tray 500 was implanted (presumably along with a side tibial component 300), the surgeon can replace the second embodiment backing tray 500 with a first embodiment backing tray 400 and a middle backing tray 1300. In either case, whereas in the previous embodiments the middle backing tray 1300 was configured to support the center tibial component 1200, a half-span tibial component 600, or a full-span tibial component 700, in this embodiment, the middle backing tray 1300 will support an alternate embodiment center tibial component 1500, which is shown in FIGS. 13A-13D.

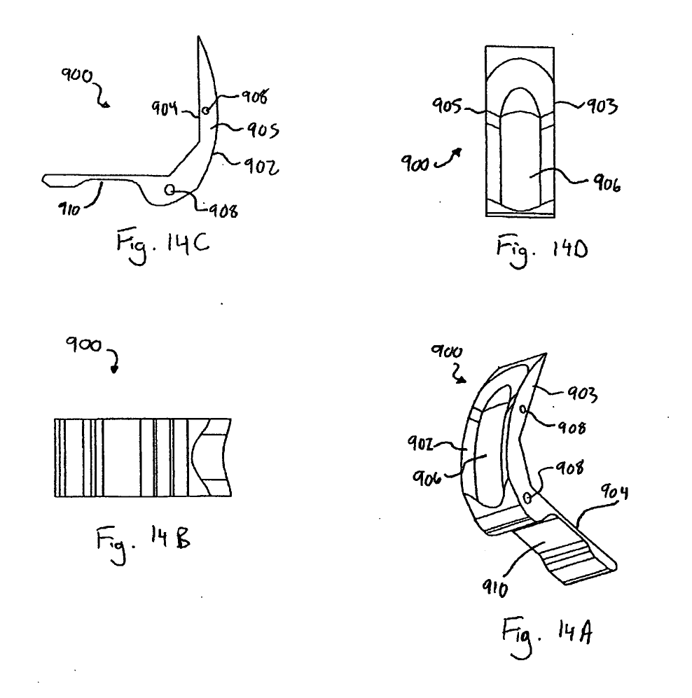

Preferably, the alternate embodiment center tibial component 1500 is formed of a strong biocompatible metal such as a cobalt-chromium alloy, a titanium alloy, or stainless steel. Additionally or alternatively, the alternate embodiment center tibial component 1500 may be formed from a strong ceramic (e.g., an alumina, zirconia, or carbon-based ceramic), one or more high performance polymers, and/or one or more high performance polymer composites (e.g., a composite material made of nano particles of PTFE and PEEK; particle ratios can be either fixed or can vary in the range on 0-100% (and vice versa), thereby enabling gradual changes in material properties in the component). Additionally or alternatively, the component may be made of a material of the types described in U.S. patent application Ser. No. 10/914,615. Of course, other biocompatible materials may be used to form the center tibial component 1500. The alternate embodiment center tibial component 1500 combines the functionality of the first embodiment center tibial component 1200 with the fin 840 of the PCL substituting device 800 and, therefore, like parts are given like reference numbers, a repetitive discussion of which will be omitted. However, the functionality of the fins 840 of the PCL substituting device 800 and the alternate embodiment center tibial component 1500 will now be discussed with respect to an alternate embodiment center femoral component 900, which is shown in FIG. 14A-14D.

The alternate center femoral component 900, which can be implanted instead of the center femoral component 200 shown in FIGS. 4A-4C, has a generally L-shape, as shown best in FIG. 14C. Preferably, the alternate center femoral component 900 is formed of a strong biocompatible metal such as a cobalt-chromium alloy, a titanium alloy, or stainless steel. Additionally or alternatively, the alternate center femoral component 900 may be formed from a strong ceramic (e.g., an alumina, zirconia, or carbon-based ceramic), one or more high performance polymers, and/or one or more high performance polymer composites (e.g., a composite material made of nano particles of PTFE and PEEK; particle ratios can be either fixed or can vary in the range on 0-100% (and vice versa), thereby enabling gradual changes in material properties in the component). Additionally or alternatively, the component may be made of a material of the types described in U.S. patent application Ser. No. 10/914,615. Of course, other biocompatible materials may be used to form the alternate center femoral component 900.

The center femoral component 900 has a front face 902 and a bone contacting face 904, which is configured to be implanted in the central region C of the femur 30. A depression 910, which is provided in the front face 902, is configured to receive an outer face 842 of the fin 840 of PCL substituting device 800 or the alternate center tibial component 1500. As a result, the outer face 842 of the fin 840 is configured to rock back-and-forth within the depression 910, thereby enabling the tibia 40 to bend with respect to the femur 30. Moreover, as a result of this mechanical movement, any cartilage in the vicinity of the center femoral component 900 and either the substituting device 800 or the alternate center tibial component 1500 is substantially protected from wear.

The alternate embodiment center femoral component 900 may have a connection mechanism on side faces 903, 905 thereof. For example, the center femoral component may have holes 908 that are sized to receive pins (which may be similar to pins 1600 shown in FIGS. 18A and 18B) projecting from an outer face 103, 105 of, e.g., a femoral component 100. Of course, other fasteners (e.g., screws, morse tapers, dovetail tenon/mortise, locking clips, etc.) may be employed. The alternate embodiment center femoral component 900 also may, like the first embodiment center femoral component 200, have a depression 906.

The depressions 206, 906 of the first and second embodiment center femoral components 200, 900 are configured to slidingly receive a patellar backing device 1000, which will now be discussed with respect to FIGS. 15A-15D. Preferably, the patellar backing device 1000 is formed of a strong biocompatible metal such as a cobalt-chromium alloy, a titanium alloy, or stainless steel. Additionally or alternatively, the patellar backing device 1000 may be formed from a strong ceramic (e.g., an alumina, zirconia, or carbon-based ceramic), one or more high performance polymers, and/or one or more high performance polymer composites (e.g., a composite material made of nano particles of PTFE and PEEK; particle ratios can be either fixed or can vary in the range on 0-100% (and vice versa), thereby enabling gradual changes in material properties in the component). Additionally or alternatively, the component may be made of a material of the types described in U.S. patent application Ser. No. 10/914,615. Of course, other biocompatible materials may be used to form the patellar backing device 1000.

The patellar backing device 1000 is a generally dome-shaped component, which is configured to be implanted in the back side of the patella 50 and reside in patellar region G. Moreover, one or more projections 1008 may extend from an underside 1004 of backing device 1000. The projections 1008 may be journalled into corresponding sized holes formed in the back of the patella 50, thereby immobilizing the patellar backing device 1000 with respect to the patella 50.

A central region 1002 of a dome 1010 portion of the patellar backing device 1000 dome may be generally flat. The flat central region 1002 is configured to be slidably received in the depressions 206, 906 of the first and second embodiment center femoral components 200, 900. As a result of the sliding nature between the flat central region 1002 of the dome 1010 and the depressions 206, 906 of the first and second embodiment center femoral components 200, 900, the patella 50 remains able to move relative to the femur 30 and the tibia 40, when the leg bends.

Another component of the present invention relates to a tibial tray post 1100, which is shown in FIGS. 16A-16D. Preferably, the tibial tray post 1100 is formed of a strong biocompatible metal such as a cobalt-chromium alloy, a titanium alloy, or stainless steel. Additionally or alternatively, the tibial tray post 1100 may be formed from a strong ceramic (e.g., an alumina, zirconia, or carbon-based ceramic), one or more high performance polymers, and/or one or more high performance polymer composites (e.g., a composite material made of nano particles of PTFE and PEEK; particle ratios can be either fixed or can vary in the range on 0-100% (and vice versa), thereby enabling gradual changes in material properties in the component). Additionally or alternatively, the component may be made of a material of the types described in U.S. patent application Ser. No. 10/914,615. Of course, other biocompatible materials may be used to form the tibial tray post 1100.

The tibial tray post 1100 is configured to be driven deep into the lateral B, central D, and/or medial F regions of the tibia 40, thereby providing support in cases where the upper end of the tibia 40 is significantly diseased and/or degenerated. The tibial tray post 1100 may be a spike, although the illustrative embodiment shows a dome portion 1110 that is not designed in a spike-like manner. The dome portion 1110 is connected to a generally cylindrically shaped portion 1130, which, in turn, is connected to a conical portion 1120. A projection 1140, which extends from an underside 1104 of the conical portion 1120, is sized to be received by the holes 414, 1314, 514 formed in the wells 410, 1310, 510 of the first embodiment backing tray 400, middle backing tray 1300, second embodiment backing tray 500, respectively. As a result, the tibial tray post 1100 can be immobilized with respect to any of the tibial trays 400, 1300, 500.

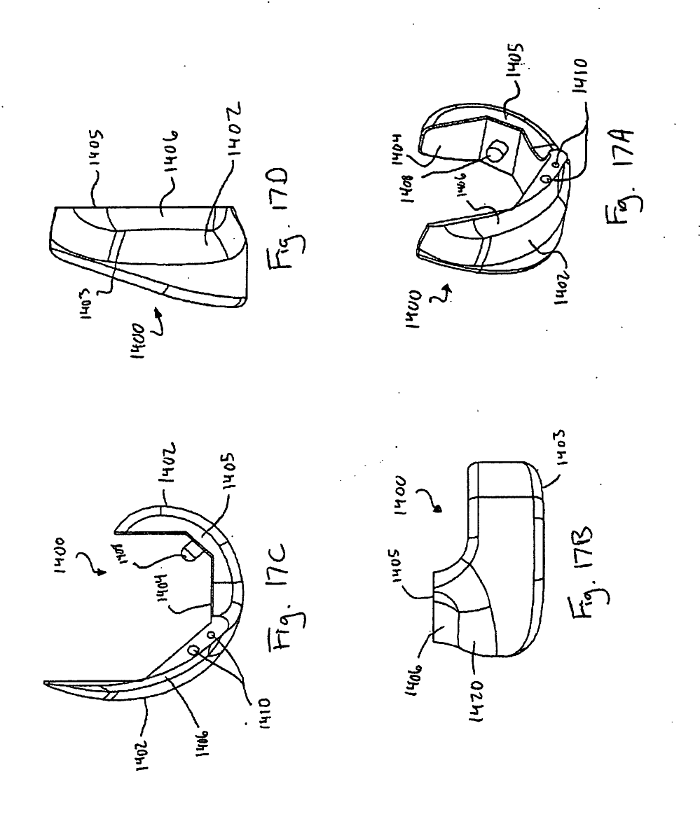

Another component of the present invention will now be discussed with respect to FIGS. 17A-17B, which show a second embodiment femoral component 1400 (half-span femoral component). Preferably, the second embodiment femoral component 1400 is formed of a strong biocompatible metal such as a cobalt-chromium alloy, a titanium alloy, or stainless steel. Additionally or alternatively, the second embodiment femoral component 1400 be formed from a strong ceramic (e.g., an alumina, zirconia, or carbon-based ceramic), one or more high performance polymers, and/or one or more high performance polymer composites (e.g., a composite material made of nano particles of PTFE and PEEK; particle ratios can be either fixed or can vary in the range on 0-100% (and vice versa), thereby enabling gradual changes in material properties in the component). Additionally or alternatively, the component may be made of a material of the types described in U.S. patent application Ser. No. 10/914,615. Of course, other biocompatible materials may be used to form the second embodiment femoral component 1400.

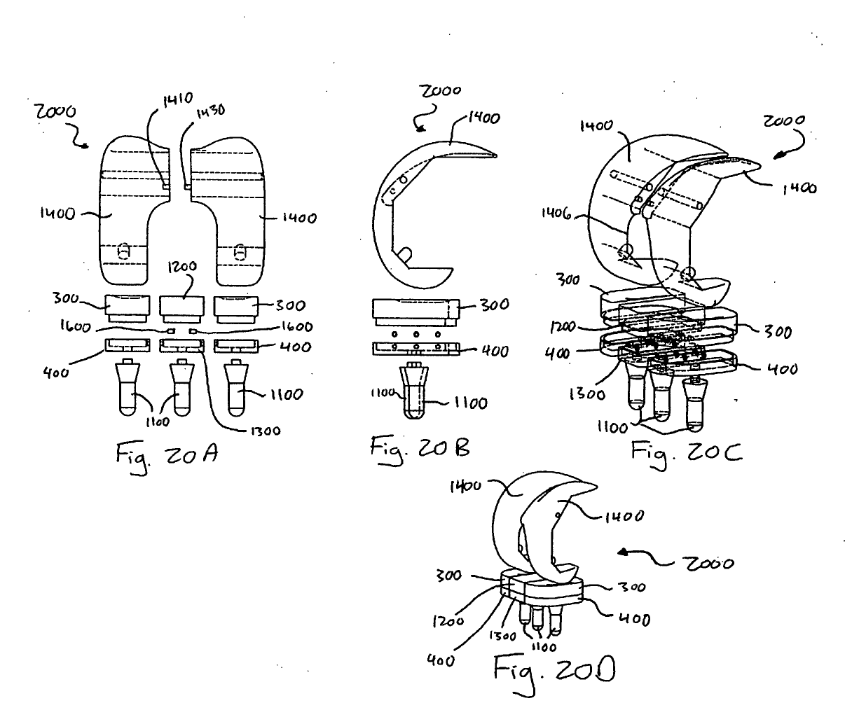

The second embodiment femoral component 1400 combines the functionality of the first embodiment femoral component 100 and one half of the first embodiment center femoral component 200. The shown embodiment of the half-span femoral component 1400 is configured to be implanted in the lateral region A of the femur 30. It should be recognized, however, that a mirror image (shown in FIGS. 20A-20D) of the half-span femoral component 1400 can also be implanted in medial femoral region E.

The second embodiment femoral component 1400 is, like the first embodiment femoral component 100, generally c-shaped and includes a front side 1402, a right face 1403, a rear side 1404, and a left face 1405. The front side 1402 is generally smooth and curved such that the front side 1402 can engage the underside of the patella 50 and the upper end of the tibia 40. The rear side 1404 includes a projection 1408 that is configured to be journalled into a corresponding hole formed (e.g., by drilling) in the femur 30; the projection 1408 thereby serves as a stabilizing unit of the component 1400, when implanted in the femur 30. The second embodiment femoral component 1400 may include a connection mechanism. In the shown embodiment, the connection mechanism includes holes 1410, which are provided on the right and left faces 1403, 1405 and which are sized to receive mating pins 1430 (shown best in FIG. 20A). Of course, other fasteners (e.g., screws, morse tapers, dovetail tenon/mortise, locking clips, etc.) may be employed.

One distinguishing feature of the second embodiment femoral component 1400, as compared to the first embodiment femoral component 100, is that the half-span femoral component 1400 includes a wide portion 1420. Moreover, the wide portion 1420 includes one half of a depression 1406, which is configured to engage a patellar backing device 1000 of the type shown in FIGS. 15A-15D. By combining the shown half-span femoral component 1400 with its mirror image component, a complete depression 1406 (shown best in FIG. 20C) can be formed and, therefore, the two second embodiment femoral components 1400 can serve the same functionality as two first embodiment femoral components 100 and a first embodiment center femoral component 200.

FIGS. 18A, 18B, and 18C are exploded front, side, and perspective views, respectively, of a prosthetic device 1800 that includes: (a) two femoral components 100 of the type shown in FIGS. 3A-3D for implantation in the lateral A and medial E regions of the femur 30; (b) a center femoral component 200 of the type shown in FIGS. 4A-4D for implantation between the femoral components 100; (c) two backing trays 300 of the type shown in FIGS. 7A-7D for implantation in the lateral B and medial F regions of the tibia 40; (d) a middle backing tray 1300 of the type shown in FIGS. 8A-8D for implantation in the central region D of tibia 40 between the backing trays 400; (e) two tibial components 300 of the type shown in FIGS. 5A-5D for implantation in the backing trays 400; (f) a center tibial component 1200 of FIGS. 6A-6D for implantation in the middle backing tray 1300; and (g) a plurality of tibial tray posts 1000 of the type shown in FIGS. 16A-16D for implantation in the lateral B, central D, and medial F regions of the tibia 40. As shown, the device 1800 may include one or more pins 1600 that are configured to be received in the holes 408, 1308 in the back walls 404 of the backing trays 400 and the longer sides 1302 of the middle backing tray 1300. Similar pins 1600 may be used to fasten other components, e.g., the femoral components 100 and the middle femoral component 200. Of course, other fasteners (e.g., screws, morse tapers, dovetail tenon/mortise, locking clips, etc.) may be employed.

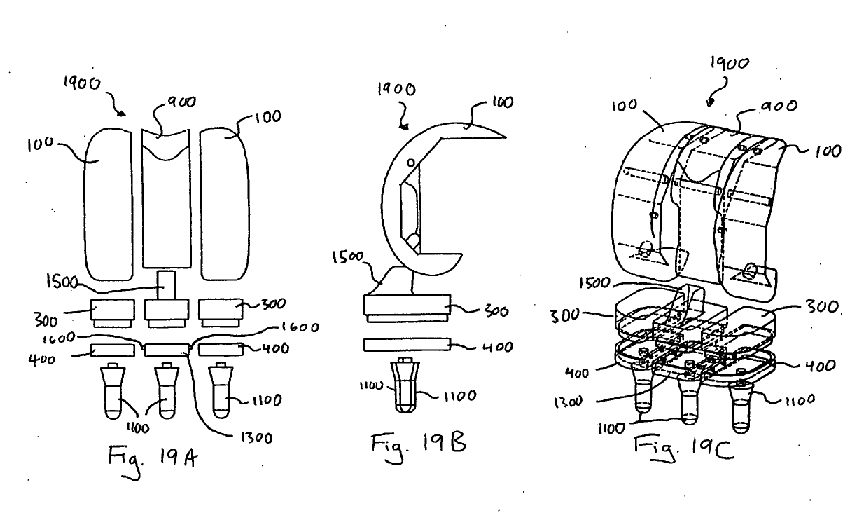

FIGS. 19A, 19B, and 19C are exploded front, side, and perspective views, respectively, of a prosthetic device 1900 that includes: (a) two femoral components 100 of the type shown in FIGS. 3A-3D for implantation in the lateral A and medial E regions of the femur 30; (b) two backing trays 400 of the type shown in FIGS. 7A-7D for implantation in the lateral B and medial F regions of the tibia 40; (c) a middle backing tray 1300 of the type shown in FIGS. 8A-8D for implantation in the central region D of the tibia 40 between the backing trays 400; (d) two tibial components 300 of the type shown in FIGS. 5A-5D for implantation in the backing trays 400; (e) a cruciate substituting center femoral component 900 of the type shown in FIGS. 13A-13D for implantation in the central region C of the femur 30 between the femoral components 100; (f) a center tibial component 1500 for implantation in the middle backing tray 1300; and (g) a plurality of tibial tray posts 1000 of the type shown in FIGS. 16A-16D for implantation in the lateral B, central D, and medial F regions of the tibia 40. As shown, the device 1900 may include one or more pins 1600 that are configured to be received in the holes 408, 1308 in the back walls 404 of the backing trays 400 and the longer sides 1302 of the middle backing tray 1300. Similar pins 1600 may be used to fasten other components, e.g., the femoral components 100 and the middle femoral component 900. Of course, other fasteners (e.g., screws, morse tapers, dovetail tenon/mortise, locking clips, etc.) may be employed.

FIGS. 20A, 20B, 20C, and 20D are exploded front, exploded side, exploded perspective, and assembled views, respectively, of a prosthetic device that includes: (a) two femoral components 1400 of the type shown in FIGS. 17A-17D for implantation in the lateral A, central C, and medial E regions of the femur 30; (b) two backing trays 400 of the type shown in FIGS. 7A-7D for implantation in the lateral B and medial F regions of the tibia 40; (c) a middle backing tray 1300 of the type shown in FIGS. 8A-8D for implantation in central region D of the tibia 40 between the backing trays 400; (d) two tibial components 300 of the type shown in FIGS. 5A-5D for implantation in the backing trays 400; (e) a center tibial component 1200 of the type shown in FIGS. 6A-6D for implantation in the middle backing tray 1300 and (f) a plurality of tibial tray posts 1000 of the type shown in FIGS. 16A-16D for implantation in the lateral B, central D, and medial F regions of the tibia 40. As shown, the device 2000 may include one or more pins 1600 that are configured to be received in the holes 408, 1308 in the back walls 404 of the backing trays 400 and the longer sides 1302 of the middle backing tray 1300. In addition, other pins 1430 may be used to fasten other components, e.g., the femoral components 1400. Of course, other fasteners (e.g., screws, morse tapers, dovetail tenon/mortise, locking clips, etc.) may be employed.

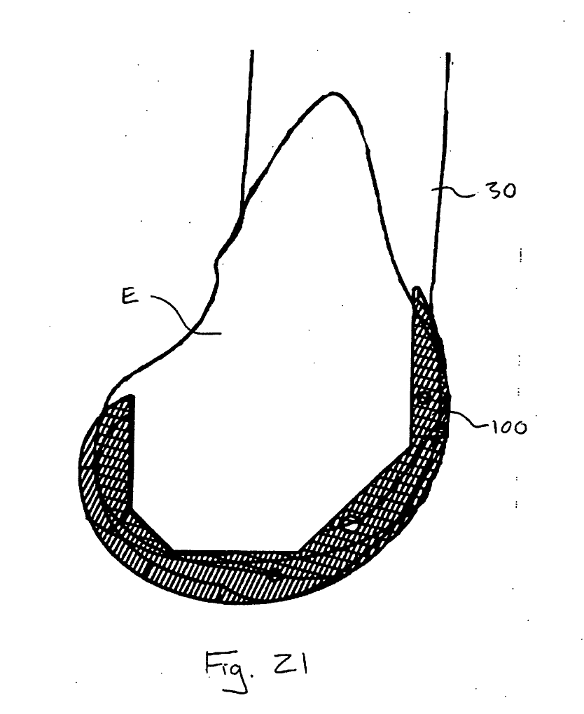

FIG. 21 is a cross-sectional view of the femur 30 with a femoral component 100 of the type shown in FIGS. 3A-3D implanted on the medial region E of the femur 30.

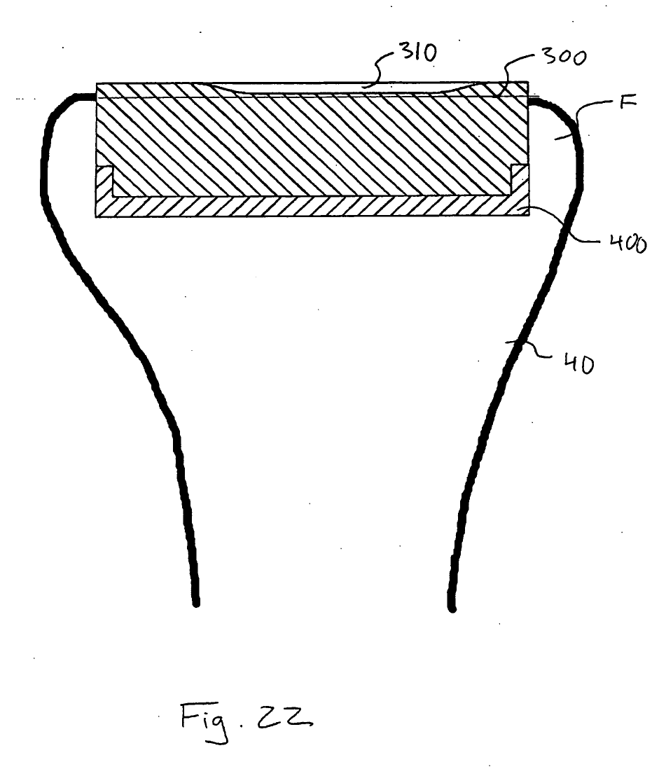

FIG. 22 is a cross-sectional view of the tibia 40 with a backing tray 400 of the type shown in FIGS. 7A-7D implanted in the medial region F of the tibia 40 and with a tibial component 300 of the type shown in FIGS. 5A-5D implanted in the backing tray 400.

FIG. 23 is a cross-sectional view of the tibia 40 with a tibial component of the type shown in FIGS. 5A-5D implanted directly on the medial region F of the tibia 40.

The present invention can provide a number of advantages, examples of which are provided below. It may be possible, however, to practice the invention without achieving some or all of the described advantages.

The present invention allows for minimally invasive surgery by limiting the number and size of incisions necessary. Specifically, in some embodiments only one incision will be necessary. Moreover, due to the size of the components, each component can be inserted into the body of a patient through an opening in the patient's skin that is no greater than approximately two inches and, in some case, less than approximately one inch. After inserting the components through the opening, the prosthetic device can be assembled within the knee cavity, if necessary, and implanted. The ability to insert the components through such a small opening in the patient's skin reduces damage to soft tissue and reduces recovery time.

The present invention also can reduce the amount of healthy bone that is displaced. If only a portion of the knee (e.g., the lateral regions A, B of the femur 30 and the tibia 40) is diseased or degenerated, the present invention can be used to address only that portion of the knee. The remainder of the knee, which is healthy, is not substantially affected. More specifically, the present invention provides the surgeon with a range of components so that he or she can select the particular components (and the particular size thereof) needed to treat only the damaged portion(s) of the knee. The following is a table of various knee conditions and the corresponding components that may be implanted to address each particular condition:

| | Affected | | |
|---|---|---|---|
| Knee | Regions of | Implant | |
| Condition | the Knee | Option | Components Implanted (Implant Location) |
| Lateral | A, B | | femoral component 100 (region A); |
| compartment | | | side tibial component 300 (region B); and |
| arthritis | | | backing tray 400 (region B) |
| Medial | E, F | | femoral component 100 (region E); |
| compartment | | | side tibial component 300 (region F); and |
| arthritis | | | backing tray 400 (region F) |
| Lateral | A, C, B, | Option 1 | femoral component 100 (region A); |
| compartment | D, G | | center femoral component 200 (region C); |
| with | | | half-span tibial component 600 (region B, D); |
| patellofemoral | | | backing tray 500 (region B, D); and |
| degeneration | | | patellar backing device 1000 (region G) |
| | | Option 2 | femoral component 100 (region A); |
| | | | center femoral component 200 (region C); |
| | | | side tibial component 300 (region B); |
| | | | backing tray 400 (region B); |
| | | | middle backing tray 1300 (region D); |
| | | | center femoral component 1200 (region D); and |
| | | | patellar backing device 1000 (region G) |
| Medial | E, C, D, F, G | Option 1 | femoral component 100 (region E); |
| compartment | | | center femoral component 200 (region C); |
| with | | | half-span tibial component 600 (region F, D); |
| patellofemoral | | | backing tray 500 (region F, D); and |
| degeneration | | | patellar backing device 1000 (region G) |
| | | Option 2 | femoral component 100 (region E); |
| | | | center femoral component 200 (region C); |
| | | | side tibial component 300 (region F); |
| | | | backing tray 400 (region F); |
| | | | middle backing tray 1300 (region D); |
| | | | center femoral component 1200 (region D); and |
| | | | patellar backing device 1000 (region G) |
| Bicompartmental | A, B, E, F, G | | femoral component 100 (region A); |
| with unaffected | | | side tibial component 300 (region B); |
| patellofemoral | | | backing tray 400 (region B); |
| compartment | | | femoral component 100 (region E); |
| | | | side tibial component 300 (region F); |
| | | | backing tray 400 (region F); and |
| | | | patellar backing device 1000 (region G) |
| Bicompartmental | A, B, D, | Option 1 | femoral component 100 (region A); |
| femur with | E, F, G | | side tibial component 300 (region B); |
| complete tibial | | | backing tray 400 (region B); |
| degeneration | | | femoral component 100 (region E); |
| | | | side tibial component 300 (region F); |
| | | | backing tray 400 (region F); |
| | | | center tibial component 1200 (region D); |
| | | | middle backing tray 1300 (region D); |
| | | | tibial tray post 1100 (region D); and |
| | | | patellar backing device 1000 (region G) |
| | | Option 2 | femoral component 100 (region A); |
| | | | half-span tibial component 600 (region B, D); |
| | | | backing tray 500 (region B, D); |
| | | | femoral component 100 (region E); |
| | | | half-span tibial component 600 (region F, D); |
| | | | backing tray 500 (region F, D); |
| | | | tibial tray post 1100 (region D); and |
| | | | patellar backing device 1000 (region G) |
| | | Option 3 | femoral component 100 (region A); |
| | | | backing tray 500 (region B, D); |
| | | | femoral component 100 (region E); |
| | | | backing tray 500 (region D, F); |
| | | | full-span tibial component 700 (region B, D, F); |
| | | | tibial tray post 1100 (region D); and |
| | | | patellar backing device 1000 (region G) |
| | | Option 4 | femoral component 100 (region A); |
| | | | backing tray 400 (region B); |
| | | | center tibial component 1300 (region D); |
| | | | femoral component 100 (region E); |
| | | | backing tray 400 (region F); |
| | | | full-span tibial component 700 (region B, D, F); |
| | | | tibial tray post 1100 (region D); and |
| | | | patellar backing device 1000 (region G) |
| Bicompartmental | A, B, C, E, | Option 1 | femoral component 100 (region A); |
| tibia with | F, G | | center femoral component 200 (region C); |
| complete | | | femoral component 100 (region E); |
| femoral | | | side tibial component 300 (region B); |
| degeneration | | | backing tray 400 (region B); |
| | | | side tibial component 300 (region F); |
| | | | backing tray 400 (region F); and |
| | | | patellar backing device 1000 (region G) |
| | | Option 2 | half-span femoral component 1400 (region A); |
| | | | half-span femoral component 1400 (region E); |
| | | | side tibial component 300 (region B); |
| | | | backing tray 400 (region B); |
| | | | side tibial component 300 (region F); |
| | | | backing tray 400 (region F); and |
| | | | patellar backing device 1000 (region G) |
| Full | A, B, C, | Option 1 | femoral component 100 (region A); |
| degeneration of | D, E, F, G | | center femoral component 200 (region C); |
| all compartments | | | femoral component 100 (region E); |
| | | | side tibial component 300 (region B); |
| | | | backing tray 400 (region B); |
| | | | side tibial component 300 (region F); |
| | | | backing tray 400 (region F); |
| | | | center tibial component 1200 (region D); |
| | | | middle backing tray 1300 (region D); |
| | | | tibial tray post 1100 (region D); and |
| | | | patellar backing device 1000 (region G) |
| | | Option 2 | femoral component 100 (region A); |
| | | | center femoral component 200 (region C); |
| | | | femoral component 100 (region E); |
| | | | patellar backing device 1000 (region G); |
| | | | half-span tibial component 600 (region B, D); |
| | | | backing tray 500 (region B, D); |
| | | | half-span tibial component 600 (region D, F); |
| | | | backing tray 500 (region D, F); and |
| | | | tibial tray post 1100 (region D) |
| | | Option 3 | femoral component 100 (region A); |
| | | | center femoral component 200 (region C); |
| | | | femoral component 100 (region E); |
| | | | patellar backing device 1000 (region G); |
| | | | full-span tibial component 700 (region B, D, F) |
| | | | backing tray 500 (region B, D); |
| | | | backing tray 500 (region D, F); and |
| | | | tibial tray post 1100 (region D) |
| | | Option 4 | half-span femoral component 1400 (region A); |
| | | | half-span femoral component 1400 (region E); |
| | | | patellar backing device 1000 (region G); |
| | | | side tibial component 300 (region B); |
| | | | backing tray 400 (region B); |
| | | | side tibial component 300 (region F); |
| | | | backing tray 400 (region F); |
| | | | center tibial component 1200 (region D); |
| | | | tibial tray post 1100 (region D); and |
| | | | middle backing tray 1300 (region D) |
| | | Option 5 | half-span femoral component 1400 (region A); |
| | | | half-span femoral component 1400 (region E); |
| | | | patellar backing device 1000 (region G); |
| | | | half-span tibial component 600 (region B, D); |
| | | | backing tray 500 (region B, D); |
| | | | half-span tibial component 600 (region D, F); |
| | | | backing tray 500 (region D, F); and |
| | | | tibial tray post 1100 (region D) |
| | | Option 6 | half-span femoral component 1400 (region A); |
| | | | half-span femoral component 1400 (region E); |
| | | | patellar backing device 1000 (region G); |
| | | | full-span tibial component 700 (region B, D, F); |
| | | | backing tray 500 (region B, D); |
| | | | backing tray 500 (region D, F); and |
| | | | tibial tray post 1100 (region D) |
| | | Option 7 | femoral component 100 (region A); |
| | | PCL | femoral component 100 (region E); |
| | | Sacrifice | patellar backing device 1000 (region G); |
| | | No. 1 | center femoral component 900 (region C); |
| | | | backing tray 500 (region B, D); |
| | | | backing tray 500 (region D, F); |
| | | | tibial tray post 1100 (region D); and |
| | | | cruciate ligament substitute 800 (region B, D, F) |
| | | Option 7 | femoral component 100 (region A); |
| | | PCL | femoral component 100 (region E); |
| | | Sacrifice | patellar backing device 1000 (region G); |
| | | No. 2 | center femoral component 900 (region C); |
| | | | side tibial component 300 (region B); |
| | | | backing tray 400 (region B); |
| | | | side tibial component 300 (region F); |
| | | | backing tray 400 (region F); and |
| | | | center tibial component 1500 (region C) |

The present invention also can be customized to meet the needs of individual patients in other respects. For example, a variety of components can be provided for each region (a variety of components for the lateral condyle region A of the femur, a variety of components for the central region C of the femur, etc.). Each of those components has particular characteristics different from the other components for the region. A surgeon can select a component for each region having the characteristics that best meet the needs of the patient. Because the surgeon can select the best component for each region, a highly customized prosthetic device will be created by combination of selected components. The tables below lists the differing characteristics of the femoral and tibial components:

| | |
|---|---|
| Characteristics of Lateral and Medial | |
| Components | Characteristics of Central Components |

Femoral Components

| | |
|---|---|
| Size-A variation in sizes for each | Depth of patella-femoral groove-A |
| component provides the ability to mix-and- | variation in the size of the center femoral |
| match between medial and lateral | component 200, 900 enables a surgeon to |
| components. For example, a “small” medial | vary the size of the depth of the track formed |
| femoral component 100 can be used in | in the patellar groove in which the center |
| conjunction with a “large” lateral femoral | femoral component 200, 900 will be |
| component 100. | implanted. |
| Condylar geometry-A variation in condylar | Anatomic coverage-The surgeon can |
| geometry provides optimum stability for each | accommodate both symmetrically shaped and |
| side. For example, some femoral | asymmetrically shaped central portions of the |
| components 100, such as those shown in | femur. Specifically, by adjusting the size |
| FIGS. 3A-3D, 17A-1D, may have curved | and/or type of the center femoral component, |
| cross-sections, which is typically called a | the surgeon can accommodate anatomical |
| “total condylar shape.” In contrast, other | variations (or symmetry) between the medial |
| femoral components (not shown) may have a | and lateral regions of the anterior femoral |
| flatter cross-section, thereby allowing more | face. |
| rotary translation. Moreover, as previously | |
| mentioned, the mix-and-match nature of the | |
| invention enables, e.g., a curved femoral | |
| component to be implanted in the medial | |
| region of the knee and a flat femoral | |
| component to be implanted in the lateral | |
| region of the knee. | |
| Augment presence-A variation in bone- | Cruciate compatibility-Based on the size |
| filling augments (e.g., metal) provides the | and/or health of the femoral groove 32 and |
| ability to fill various sizes holes and/or gaps | the PCL, the surgeon can decide whether to |
| around the prosthesis, thereby reducing the | spare or sacrifice the PCL. In other words, |
| likelihood of infection. Typically, these | the surgeon can decide whether to use a |
| augments are affixed to a component during | center femoral component 200 or a center |
| implantation with, for example, screws, | femoral component 900. |
| expandable rivets, etc. | |
| Replaceability-As a result of the modularity | |
| of the current invention, if one femoral | |
| component becomes loose over time, only | |
| that loose component needs to be replaced or | |
| fixed, i.e., the remainder of the implant can | |
| be largely unaffected. | |

Tibial Components

| | |
|---|---|
| Size-A variation in sizes for each | Cruciate compatibility-Based on the size |
| component provides the ability to mix-and- | and/or health of the femoral groove 32 and |
| match between medial and lateral | the PCL, the surgeon can decide whether to |
| components. For example, a “small” medial | spare or sacrifice the PCL. In other words, |
| side support 300 can be used in conjunction | the surgeon can decide whether to use a PCL |
| with a “large” lateral side support 300. | substituting device 800 or a center tibial |
| | component 1500. |
| Condylar geometry-A variation in condylar | Stem or cruciform interchangeability-By |
| geometry provides optimum stability for each | providing holes 414, 1314, and 514 in the |
| side. For example, a combination of two | various backing trays 400, 1300, 500, the |
| backing trays 400 and a middle backing tray | system enables tibial tray posts 1100 (or |
| 1300 may form a generally oval shaped | other stems and/or cruciforms) to be driven |
| implant. In contrast, a combination of two | into the tibia and secured to the implant. |
| backing trays 500 may have a notch formed | Moreover, the size of the tibial tray post 1100 |
| in one side thereof (as shown in FIGS. 9A-9D). | can be selected based on a particular |
| | condition (e.g., location, disease/degeneration |
| | state, etc.) of the patient. As the projection |
| | 1140 can be generally uniform, any chosen |
| | tibial tray post 1100 can work with the |
| | selected backing trays 400, 1300, 500. |
| Augment presence-A variation in bone- | |
| filling augments (e.g., metal) provides the | |
| ability to fill various sizes holes and/or gaps | |
| around the prosthesis, thereby reducing the | |
| likelihood of infection. Typically, these | |
| augments are affixed to a component during | |
| implantation with, for example, screws, | |
| expandable rivets, etc. | |
| Replaceability-As a result of the modularity | |
| of the current invention, if one tibial | |
| component becomes loose over time, only | |
| that loose component needs to be replaced or | |
| fixed, i.e., the remainder of the implant can | |
| be largely unaffected. | |
| Thickness-A variation in polyethylene | |
| thickness enables a surgeon to vary and | |
| adjust the thickness of various components | |
| between the medial and lateral regions. This | |
| factor is particularly beneficial because it can | |
| enable a surgeon to address the so-called | |
| joint line (center of rotation) of the knee for | |
| each patient. For example, to correct a | |
| “knock-kneed” condition, the surgeon may | |
| employ a 10 mm UHMWPE inserted in the | |
| lateral portion of the tibia and a 15 mm | |
| UHMWPE insert in the medial portion of the | |
| tibia, thereby straightening the patient's gait. | |
| Bearing surfaces-A variation in components | |
| enables a surgeon to provide conventional | |
| mobile bearing inserts (not shown) in one of | |
| the medial or lateral backing trays or in both | |
| the medial and lateral trays. Mobile bearing | |
| inserts are designed such that when the knee | |
| bends, the inserts enable normal flexion- | |
| extension but also slide within the backing | |
| trays to increase rotation and translatory | |
| motion. | |

The present invention also can be configured to accommodate later surgical procedures. For example, if the attachment of a component to the bone becomes loose, only that component needs to be replaced in a later surgical procedure. The remaining “good” component(s) can be left undisturbed. Moreover, if there is an increase in the diseased or damaged area of the knee, the already implanted component(s) need not be disturbed. Additional components can be added to the knee to address the newly diseased or damaged areas of the knee.

For example, if a side femoral component 100, a backing tray 400, and a side tibial component 300 were previously implanted as a prosthetic device in the lateral side of the knee to address prior knee damage, that prosthetic device may be left intact while, during a later procedure to address subsequent knee damage to the center portion of the knee, a middle backing tray 1300 and a center tibial component 1500 are implanted. The middle backing tray 1300 may, at that time, be connected (e.g., screws, morse tapers, dovetail tenon/mortise, locking clips, etc.) to the previously implanted backing tray 400, thereby creating a second, enhanced prosthetic device.

As further example, if at the time the middle backing tray 1300 is implanted or if at a later time, the medial part of the knee is diseased or degenerated, further corrective steps may be taken. Specifically, another femoral component 100, backing tray 400, and side tibial component 300 could be implanted in the medial side of the knee, while leaving the second, enhanced prosthetic device intact. The latter implanted backing tray 400 could be connected (e.g., screws, morse tapers, dovetail tenon/mortise, locking clips, etc.) to the middle backing tray 1300, thereby creating a further enhanced, third prosthetic device. Alternatively, a full-span tibial component 700 could be attached to the backing trays 400, 1300, 400, thereby replacing the two side tibial components 300 and the center tibial component 1500.

As yet another example, in a case in which the central part of the knee becomes diseased or degenerated after either the lateral or medial side of the knee has been replaced, a middle backing tray 1300 can be added to a previously implanted backing tray 400 or a previously implanted backing tray 500 can be replaced with a combination of a backing tray 400 and a middle backing tray 1300.

The present invention is not intended to be limited to the previously described embodiments. It will be apparent to those skilled in the art that various modifications and variations can be made to the disclosed embodiments of the present invention, without departing from the scope or spirit of the invention. Accordingly, these modifications and variations are fully within the scope of the claimed invention. Therefore, it should be understood that the apparatuses and methods described herein are illustrative only and are not limiting upon the scope of the invention, which is indicated by the following claims.

What is claimed is:

1. A method of implanting a prosthetic device comprising the steps of:
selecting a first side femoral component configured to be implanted on at least one of a lateral condyle and a medial condyle of a femur;
implanting the first side femoral component on one of the lateral condyle and the medial condyle of the femur;
selecting a second side femoral component configured to be implanted on at least the other of the lateral condyle and the medial condyle of the femur, wherein the second side femoral component is selected from a plurality of femoral components configured to be used with the first side femoral component based on characteristics of the second side femoral component; and
implanting the second side femoral component on the femur.

2. The method of claim 1, wherein the characteristics of the second side femoral component include at least one of size, condylar geometry, material, augment presence, and replaceability.

3. The method of claim 1, wherein the step of selecting the first side femoral component includes selecting from a plurality of femoral components configured to be used with the second side femoral component based on characteristics of the first side femoral component.

4. The method of claim 3, wherein the characteristics of the first side femoral component include at least one of size, condylar geometry, material, material, augment presence, and replaceability.

5. The method of claim 1, wherein the steps of implanting include inserting the first and second side femoral components through an opening in a patient's skin of no greater than approximately two inches.

6. The method of claim 1, further comprising the step of:
connecting the first side femoral component to the second side femoral component.

7. The method of claim 1, further comprising the steps of:
selecting a center femoral component configured to be implanted on a central region of the femur, wherein the center femoral component is selected from a plurality of femoral components configured to be used with the first and second side femoral components based on characteristics of the center femoral component; and
implanting the center femoral component on the femur.

8. The method of claim 7, wherein the characteristics of the center femoral component include at least one of depth of patella-femoral groove, anatomic coverage, and cruciate compatibility.

9. The method of claim 7, wherein the step of implanting includes inserting the center femoral component through an opening in a patient's skin of no greater than approximately two inches.

10. The method of claim 7, further comprising the steps of:
connecting the first side femoral component to the center femoral component; and
connecting the center femoral component to the second side femoral component.

11. A method of implanting a prosthetic device comprising the steps of:
selecting a first side femoral component configured to be implanted on at least one of a lateral condyle and a medial condyle of a femur;
implanting the first side femoral component on one of the lateral condyle and the medial condyle of the femur;
selecting a center femoral component configured to be implanted on a central region of the femur, wherein the center femoral component is selected from a plurality of femoral components configured to be used with the first side femoral component based on characteristics of the center femoral component; and
implanting the center femoral component on the femur.

12. The method of claim 11, wherein the characteristics of the center femoral component include at least one of depth of patella-femoral groove, anatomic coverage, and cruciate compatibility.

13. The method of claim 11, wherein the step of selecting the first side femoral component includes selecting from a plurality of femoral components configured to be used with the center femoral component based on characteristics of the first side femoral component.

14. The method of claim 13, wherein the characteristics of the first side femoral component include at least one of size, condylar geometry, material, augment presence, and replaceability.

15. The method of claim 11, wherein the steps of implanting include inserting the first side and center femoral components through an opening in a patient's skin of no greater than approximately two inches.

16. The method of claim 11, further comprising the step of:
connecting the first side femoral component to the center femoral component.

17. A method of implanting a prosthetic device comprising the steps of:
selecting a first side tibial component configured to be implanted on at least one of a lateral region and a medial region of a tibia;
implanting the first side tibial component on one of the lateral region and the medial region of the tibia;
selecting a second side tibial component configured to be implanted on at least the other of the lateral region and the medial region of the tibia, wherein the second side tibial component is selected from a plurality of tibial components configured to be used with the first side tibial component based on characteristics of the second side tibial component; and
implanting the second side tibial component on the tibia.

18. The method of claim 17, wherein the characteristics of the second side tibial component include at least one of size, condylar geometry, material, augment presence, thickness, and number of bearing surfaces.

19. The method of claim 17, wherein the step of selecting the first side tibial component includes selecting from a plurality of tibial components configured to be used with the second side tibial component based on characteristics of the first side tibial component.

20. The method of claim 19, wherein the characteristics of the first side tibial component include at least one of size, condylar geometry, material, augment presence, thickness, and number of bearing surfaces.

21. The method of claim 17, wherein the steps of implanting include inserting the first and second side tibial components through an opening in a patient's skin of no greater than approximately two inches.

22. The method of claim 17, further comprising the steps of:
connecting the first side tibial component to the second side tibial component.

23. The method of claim 17, further comprising the steps of:
selecting a center tibial component configured to be implanted on a central region of the tibia, wherein the center tibial component is selected from a plurality of tibial components configured to be used with the first and second side tibial components based on characteristics of the center tibial component; and
implanting the center tibial component on the tibia.

24. The method of claim 23, wherein the characteristics of the center tibial component include at least one of cruciate compatibility, stem interchangeability, and cruciform interchangeability.

25. The method of claim 23, wherein the step of implanting includes inserting the center tibial component through an opening in a patient's skin of no greater than approximately two inches.

26. The method of claim 23, further comprising the steps of:
connecting the first side tibial component to the center tibial component; and
connecting the center tibial component to the second side tibial component.

27. A method of implanting a prosthetic device comprising the steps of:
selecting a first side tibial component configured to be implanted on at least one of a lateral region and a medial region of a tibia;
implanting the first side tibial component on one of the lateral region and the medial region of the tibia;
selecting a center tibial component configured to be implanted on a central region of the tibia, wherein the center tibial component is selected from a plurality of tibial components configured to be used with the first side tibial component based on characteristics of the center tibial component; and
implanting the center tibial component on the tibia.

28. The method of claim 27, wherein the characteristics of the center tibial component include at least one of cruciate compatibility, stem interchangeability, and cruciform interchangeability.

29. The method of claim 27, wherein the step of selecting the first side tibial component includes selecting from a plurality of tibial components configured to be used with the center tibial component based on characteristics of the first side tibial component.

30. The method of claim 29, wherein the characteristics of the first side tibial component include at least one of size, condylar geometry, material, augment presence, thickness, and number of bearing surfaces.

31. The method of claim 27, wherein the steps of implanting include inserting the first side and center tibial components through an opening in a patient's skin of no greater than approximately two inches.

32. The method of claim 27, further comprising the step of:
connecting the first side tibial component to the center tibial component.

33. A prosthetic device comprising:
a first side femoral component configured to be implanted on at least one of a lateral condyle and a medial condyle of a femur;
a second side femoral component configured to be implanted on at least the other of the lateral condyle and the medial condyle of the femur, wherein the second side femoral component is selected from a plurality of femoral components configured to be used with the first side femoral component based on characteristics of the second side femoral component.

34. The prosthetic device of claim 33, wherein the characteristics of the second side femoral component include at least one of size, condylar geometry, material, augment presence, and replaceability.

35. The prosthetic device of claim 33, wherein the first side femoral component is selected from a plurality of femoral components configured to be used with the second side femoral component based on characteristics of the first side femoral component.

36. The prosthetic device of claim 35, wherein the characteristics of the first side femoral component include at least one of size, condylar geometry, material, augment presence, and replaceability.

37. The prosthetic device of claim 33, wherein each of the femoral components is configured to be implanted in a patient by insertion through an opening in the patient's skin of no greater than approximately two inches.

38. The prosthetic device of claim 33, further comprising:
a mechanism for connecting the first side femoral component to the second side femoral component.

39. The prosthetic device of claim 33, further comprising:
a center femoral component configured to be implanted on a central region of the femur, wherein the center femoral component is selected from a plurality of femoral components configured to be used with the first and second side femoral components based on characteristics of the center femoral component.

40. The prosthetic device of claim 39, wherein the characteristics of the center femoral component include at least one of depth of patella-femoral groove, anatomic coverage, and cruciate compatibility.

41. The prosthetic device of claim 39, wherein each of the femoral components is configured to be implanted in a patient by insertion through an opening in the patient's skin of no greater than approximately two inches.

42. The prosthetic device of claim 39, further comprising:
a mechanism for connecting the first side femoral component to the center femoral component; and
a mechanism for connecting the center femoral component to the second side femoral component.

43. A prosthetic device comprising:
a first side femoral component configured to be implanted on at least one of a lateral condyle and a medial condyle of a femur; and
a center femoral component configured to be implanted on a central region of the femur, wherein the center femoral component is selected from a plurality of femoral components configured to be used with the first side femoral component based on characteristics of the center femoral component.

44. The prosthetic device of claim 43, wherein the characteristics of the center femoral component include at least one of depth of patella-femoral groove, anatomic coverage, and cruciate compatibility.

45. The prosthetic device of claim 43, wherein the first side femoral component is selected from a plurality of femoral components configured to be used with the center femoral component based on characteristics of the first side femoral component.

46. The prosthetic device of claim 45, wherein the characteristics of the first side femoral component include at least one of size, condylar geometry, material, augment presence, and replaceability.

47. The prosthetic device of claim 43, wherein each of the femoral components is configured to be implanted in a patient by insertion through an opening in the patient's skin of no greater than approximately two inches.

48. The prosthetic device of claim 43, further comprising:
a mechanism for connecting the first side femoral component to the center femoral component.

49. A prosthetic device comprising:
a first side tibial component configured to be implanted on at least one of a lateral region and a medial region of a tibia; and
a second side tibial component configured to be implanted on at least the other of the lateral region and the medial region of the tibia.

50. The prosthetic device of claim 49, wherein the first side tibial component is selected from a plurality of tibial components configured to be used with the second side tibial component based on characteristics of the first side tibial component.

51. The prosthetic device of claim 49, wherein the characteristics of the first side tibial component include at least one of size, condylar geometry, material, augment presence, thickness, and number of bearing surfaces.

52. The prosthetic device of claim 49, wherein the second side tibial component is selected from a plurality of tibial components configured to be used with the first side tibial component based on characteristics of the second side tibial component.

53. The prosthetic device of claim 52, wherein the characteristics of the second side tibial component include at least one of size, condylar geometry, material, augment presence, thickness, and number of bearing surfaces.

54. The prosthetic device of claim 49, wherein each of the tibial components is configured to be implanted in a patient by insertion through an opening in the patient's skin of no greater than approximately two inches.

55. The prosthetic device of claim 49, further comprising:
a mechanism for connecting the first side tibial component to the second side tibial component.

56. The prosthetic device of claim 49, wherein each of the tibial components includes a backing tray and a contact member.

57. The prosthetic device of claim 49, further comprising:
a center tibial component configured to be implanted on a central region of the tibia.

58. The prosthetic device of claim 57, wherein the center tibial component is selected from a plurality of tibial components configured to be used with the first and second side tibial components based on characteristics of the center tibial component.

59. The prosthetic device of claim 58, wherein the characteristics of the center tibial component include at least one of cruciate compatibility, stem interchangeability, and cruciform interchangeability.

60. The prosthetic device of claim 57, wherein each of the tibial components is configured to be implanted in a patient by insertion through an opening in the patient's skin of no greater than approximately two inches.

61. The prosthetic device of claim 57, wherein each of the tibial components includes a backing tray and a contact member.

62. The prosthetic device of claim 57, further comprising:
a mechanism for connecting the first side tibial component to the center tibial component; and
a mechanism for connecting the center tibial component to the second side tibial component.

63. A prosthetic device comprising:
a first side tibial component configured to be implanted on at least one of a lateral region and a medial region of a tibia; and
a center tibial component configured to be implanted on a central region of the tibia, wherein the center tibial component is selected from a plurality of tibial components configured to be used with the first side tibial component based on characteristics of the center tibial component.

64. The prosthetic device of claim 63, wherein the characteristics of the center tibial component include at least one of cruciate compatibility, stem interchangeability, and cruciform interchangeability.

65. The prosthetic device of claim 63, wherein the first side tibial component is selected from a plurality of tibial components configured to be used with the center tibial component based on characteristics of the first side tibial component.

66. The prosthetic device of claim 65, wherein the characteristics of the first side tibial component include at least one of size, condylar geometry, material, augment presence, thickness, and number of bearing surfaces.

67. The prosthetic device of claim 63, wherein each of the tibial components is configured to be implanted in a patient by insertion through an opening in the patient's skin of no greater than approximately two inches.

68. The prosthetic device of claim 63, further comprising:
a mechanism for connecting the first side tibial component to the center tibial component.

69. The prosthetic device of claim 63, wherein each of the tibial components includes a backing tray and a contact member.

70. A collection of components for forming a prosthetic device, comprising:
a plurality of first side femoral components configured to be implanted on at least one of a lateral condyle and a medial condyle of a femur and having different characteristics; and
a plurality of second side femoral component configured to be implanted on at least the other of the lateral condyle and the medial condyle of the femur and having different characteristics, wherein the second side femoral components can be used with the first side femoral components.

71. The collection of claim 70, wherein the characteristics of the first side femoral component include at least one of size, condylar geometry, material, augment presence, and replaceability.

72. The collection of claim 70, wherein the characteristics of the second side femoral component include at least one of size, condylar geometry, material, augment presence, and replaceability.

73. The collection of claim 70, wherein each of the femoral components is configured to be implanted in a patient by insertion through an opening in the patient's skin of no greater than approximately two inches.

74. The collection of claim 70, further comprising:
a plurality of center femoral components configured to be implanted on a central region of the femur and having different characteristics, wherein the center femoral components can be used with the first and second side femoral components.

75. The collection of claim 74, wherein the characteristics of the center femoral component include at least one of depth of patella-femoral groove, anatomic coverage, and cruciate compatibility.

76. The collection of claim 74, wherein each of the femoral components is configured to be implanted in a patient by insertion through an opening in the patient's skin of no greater than approximately two inches.

77. A collection of components for forming a prosthetic device comprising:
a plurality of first side femoral components configured to be implanted on at least one of a lateral condyle and a medial condyle of a femur and having different characteristics; and
a plurality of center femoral components configured to be implanted on a central region of the femur and having different characteristics, wherein the center femoral components can be used with the first side femoral components.

78. The collection of claim 77, wherein the characteristics of the first side femoral component include at least one of size, condylar geometry, material, augment presence, and replaceability.

79. The collection of claim 77, wherein the characteristics of the center femoral component include at least one of depth of patella-femoral groove, anatomic coverage, and cruciate compatibility.

80. The collection of claim 77, wherein each of the femoral components is configured to be implanted in a patient by insertion through an opening in the patient's skin of no greater than approximately two inches.

81. The collection of claim 77, further comprising:
a plurality of first side tibial components configured to be implanted on at least one of a lateral region and a medial region of a tibia and having different characteristics; and
a plurality of second side tibial component configured to be implanted on at least the other of the lateral region and the medial region of the tibia and having different characteristics, wherein the second side tibial components can be used with the first side tibial components.

82. The collection of claim 81, wherein the characteristics of the first side tibial component include at least one of size, condylar geometry, material, augment presence, thickness, and number of bearing surfaces.

83. The collection of claim 81, wherein the characteristics of the second side tibial component include at least one of size, condylar geometry, material, augment presence, thickness, and number of bearing surfaces.

84. The collection of claim 81, wherein each of the tibial components is configured to be implanted in a patient by insertion through an opening in the patient's skin of no greater than approximately two inches.

85. The collection of claim 81, further comprising:
a plurality of center tibial components configured to be implanted on a central region of the tibia and having different characteristics, wherein the center tibial components can be used with the first and second side tibial components.

86. The collection of claim 85, wherein the characteristics of the center tibial component include at least one of cruciate compatibility, stem interchangeability, and cruciform interchangeability.

87. The collection of claim 85, wherein each of the tibial components is configured to be implanted in a patient by insertion through an opening in the patient's skin of no greater than approximately two inches.

88. A collection of components for forming a prosthetic device, comprising:
a plurality of first side tibial components configured to be implanted on at least one of a lateral region and a medial region of a tibia and having different characteristics; and
a plurality of second side tibial component configured to be implanted on at least the other of the lateral region and the medial region of the tibia and having different characteristics, wherein the second side tibial components can be used with the first side tibial components.

89. The collection of claim 88, wherein the characteristics of the first side tibial component include at least one of size, condylar geometry, material, augment presence, thickness, and number of bearing surfaces.

90. The collection of claim 88, wherein the characteristics of the second side tibial component include at least one of size, condylar geometry, material, augment presence, thickness, and number of bearing surfaces.

91. The collection of claim 88, wherein each of the tibial components is configured to be implanted in a patient by insertion through an opening in the patient's skin of no greater than approximately two inches.

92. The collection of claim 88, further comprising:
a plurality of center tibial components configured to be implanted on a central region of the tibia and having different characteristics, wherein the center tibial components can be used with the first and second side tibial components.

93. The collection of claim 92, wherein the characteristics of the center tibial component include at least one of cruciate compatibility, stem interchangeability, and cruciform interchangeability.

94. The collection of claim 92, wherein each of the tibial components is configured to be implanted in a patient by insertion through an opening in the patient's skin of no greater than approximately two inches.

95. The collection of claim 88, further comprising:
a tibial tray post configured to be connected to at least one of the first and second side tibial components.

96. A collection of components for forming a prosthetic device comprising:
a plurality of first side tibial components configured to be implanted on at least one of a lateral region and a medial region of a tibia and having different characteristics; and
a plurality of center tibial components configured to be implanted on a central region of the tibia and having different characteristics, wherein the center tibial components can be used with the first side tibial components.

97. The collection of claim 96, wherein the characteristics of the first side tibial component include at least one of size, condylar geometry, material, augment presence, thickness, and number of bearing surfaces.

98. The collection of claim 96, wherein the characteristics of the center tibial component include at least one of cruciate compatibility, stem interchangeability, and cruciform interchangeability.

99. The collection of claim 96, wherein each of the tibial components is configured to be implanted in a patient by insertion through an opening in the patient's skin of no greater than approximately two inches.

100. The collection of claim 96, further comprising:
a tibial tray post configured to be connected to at least one of the first side and center tibial components.

101. A method of implanting a prosthetic device comprising the steps of:
evaluating a knee of a patient including a previously implanted prosthetic device;
implanting in the knee an additional component of a prosthetic device adjacent the previously implanted prosthetic device, while maintaining in the knee at least a portion of the previously implanted prosthetic device; and
attaching the additional component to the maintained portion of the previously implanted prosthetic device.
